SEQUENCE LISTING (2 sequences)

SEQ ID NO: 1 - protein, 8 aa, Artificial Sequence
  Ova peptide
SIINFEKL

SEQ ID NO: 2 - protein, 15 aa, Artificial Sequence
  Amino acid tag for protein biotinylation
GLNDIFEAQKIEWHE

(19) United States
(12) Patent Application Publication
(10) Pub. No.: US 2010/0151031 A1
(43) Pub. Date: Jun. 17, 2010

(54) DISCRETE SIZE AND SHAPE SPECIFIC ORGANIC NANOPARTICLES DESIGNED TO ELICIT AN IMMUNE RESPONSE

(76) Inventors: Joseph M. DeSimone, Chapel Hill, NC (US); Robby Petros, Chapel Hill, NC (US); Jeffrey Frelinger, Chapel Hill, NC (US); Adam Buntzman, Holly Springs, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/531,892

(22) PCT Filed: Mar. 24, 2008
(86) PCT No.: PCT/US08/58022

§ 371 Date: Jan. 25, 2010

Related U.S. Application Data
(60) Provisional application No. 60/919,720, filed on Mar. 23, 2007.

(51) Int. Cl. A61K 9/14; A61K 39/00; A61K 39/08; A61K 39/145; A61K 39/21; A61K 39/29; A61K 39/02; A61K 39/12; A61K 39/23; A61K 39/395; A61P 37/04

(52) U.S. Cl. 424/489; 424/184.1; 424/239.1; 424/209.1; 424/208.1; 424/227.1; 424/228.1; 424/236.1; 424/204.1; 424/207.1; 424/233.1; 424/277.1; 424/173.1

(57) ABSTRACT

The presently disclosed invention is broadly directed to therapeutic micro- and/or nanoparticles designed to target an immune cell with an active agent. More particularly, the particles have a predetermined geometry and a broadest dimension of less than about 10 μm. The immune cell-targeted micro and/or nanoparticles may additionally comprise a biocompatible polymer.

### FEDERALLY SPONSORED RESEARCH OR DEVELOPMENT

This invention was made with U.S. Government support from the National Institutes of Health under Grant No. AI 52435 and the Defense Advanced Research Projects Agency of the U.S. Department of Defense under Grant No. W911NF-06-1-0343. Thus, the U.S. Government has certain rights in the invention.

### TECHNICAL FIELD OF THE INVENTION

Generally, the presently disclosed invention relates to therapeutic nanoparticles. More particularly, the therapeutic nanoparticles are size and shape specific and have a composition that includes an immune cell targeting component.

### BACKGROUND OF THE INVENTION

Immune responses are the product of a complex process of antigen uptake and presentation to T and B lymphocytes. Critical in this process is the delivery of antigen to antigen presenting cells (APCs). While the details of antigen presentation have yet to be entirely unraveled, it is clear that soluble proteins are taken up APCs by a combination of micro-pinocytosis and receptor mediated endocytosis, often using receptors such as the mannose receptor. The number of APCs is small, particularly the highly efficient dendritic cells.

Many of the methods that enhance antigen presentation function by activating APCs via cell surface receptors, including Toll-like receptors and CD40. This includes adjuvants that slow the release of antigen and at the same time activate antigen presenting cells (e.g., Freund's complete adjuvant). However, these methods of enhanced antigen presentation, particularly the use of adjuvants, produce a number of undesirable side effects, including fever, malaise and hyperactivation of the immune system. Thus, a need remains for enhancing the uptake and presentation of antigens without undesirable side effects.

### SUMMARY OF THE INVENTION

Compositions and methods for the delivery of active agents to an immune cell are provided. The compositions include a plurality of monodisperse micro and/or nanoparticles, the particles having a predetermined geometry and a broadest dimension of less than about 10 μm, where the particles comprise an active agent and an immune cell targeting component. The immune cell-targeted micro and/or nanoparticles may additionally comprise a biocompatible polymer. In some embodiments, the particles are formed by the biocompatible polymer. The compositions are useful for the delivery of any active agent, including proteins, small molecules, pharmaceuticals, nucleotide sequences such as DNA, RNA, and siRNA, imaging agents, and the like specifically to immune cells. In some embodiments, the immune cell is an APC, and the compositions (including, e.g., an immunogenic component) are useful for eliciting an immune response in a subject.

Using methods of the invention, immune cell-targeted micro and/or nanoparticles can be formulated into a discrete size and shape. These particles can be formulated into pharmaceutical compositions containing an active agent and an immune cell targeting component for administration to a subject in need thereof.

### BRIEF DESCRIPTION OF THE DRAWINGS

FIG. 1 is a series of DIC (left), fluorescence (center) and SEM (right) images of dox-containing PEG/HDT 2×2×1 μm particles.

FIG. 2 is a series of SEM images of PEG/HDT 2×2×1 μm particles surface functionalized with carbonyldiimidazole.

FIG. 3A is a graph illustrating the determination of surface streptavidin (Alexa Fluor 647) concentration of PRINT™ particles by titration with biotin-4-fluorescein.

FIG. 3B is a graph illustrating the determination of surface biotin-rat IgG2b isotype control antibody concentration on PRINT™ particles by titration with biotin-rat IgG2b isotype control.

FIG. 4 is a series of SEM images of cylindrical PRINT™ particles (200 nm diameter×200 nm height) that have anti-mouse CD 11b (A), anti-mouse CD11c (B), anti-mouse CD80 (C), and rat IgG2b (D) conjugated to the surface.

FIG. 5 is a graph showing the selective targeting of PRINT™ particles to either CD11b or CD11 c expressing antigen presenting cells in vitro.

FIG. 6 is a bar graph showing OTII spleenocyte proliferation after treatment with PRINT™ particles. OVA is a peptide from ovalbumin. The cells are transgenic for a T cell receptor that recognizes ova, so the cells treated with OVA are positive control samples. BDC is a peptide from a diabetes antigen called BDC2.5. The cells should not recognize the BDC antigen, so cells treated with BDC represent negative controls.

FIG. 7 is a graph showing the effect of PRINT™ particles on the proliferative response of CD4 T cells to ovalbumin.

FIG. 8A is a schematic depiction of the process for attaching targeting ligands to the surface of PRINT™ particles. The scheme shows a pre-PRINT™ strategy where PEG₄₈₅-monomethacrylate is reacted with CDI prior to PRINT™ particle formation.

FIG. 8B is a schematic depiction of the process for attaching targeting ligands to the surface of PRINT™ particles. The scheme shows a post-PRINT™ strategy where PEG₄₈₅-monomethacrylate is incorporated into the particle and then reacted with CDI.

FIG. 9 is a series of SEM images of cylindrical PRINT™ particles (200 nm diameter×200 nm height) that have a carbonylimidazole functionalized surface.

FIG. 10 is an SEM image of cylindrical PRINT™ particles (200 nm diameter×200 nm height) that have primary amine groups on their surface (left), and a scheme for installing NP groups on the surface of these particles using TNBS (right).

FIG. 11 is a schematic drawing of disulfide (left) and acetal (right) cross-linking monomers used to prepare degradable PRINT™ particles.

FIG. 12 is a scheme for attachment of streptavidin (Alexa Fluor 647) to PRINT™ particles prepared using a degradable disulfide cross-linker.

FIG. 13 is a series of SEM images (top three) of rectangular 2×2×1 μm PRINT™ particles made using a degradable disulfide cross-linker and then reacted with CDI. DIC (bottom left) and fluorescence (bottom right) images of the same PRINT™ particles after reaction with streptavidin (Alexa Fluor 647) are also shown. Fluorescence signal demonstrates the successful attachment of streptavidin (Alexa Fluor 647) to the particle surface.

FIG. 14 shows fluorescence intensity of pro-drug, avidinated, dox-loaded PRINT particles stirred in buffered solutions at a pH of 5.0 or 7.4, as monitored by flow cytometry.

FIG. 15 shows cellular uptake by Sup-B8 cancer cells (A) of PRINT particles coated with a matched peptide targeting ligand, and no cellular uptake by Ramos cells (B) of PRINT particles coated with a mismatched peptide targeting ligand.

FIG. 16 shows cell viability of Sup-B8 cancer cells (A) as a function of PRINT particle dosing, and cell viability of Ramos cells (B) as a function of PRINT particle dosing.

FIG. 17 shows T cell killing as a function of PRINT particle dosing, transgenic mouse model and targeting p-MHC ligand.

### DETAILED DESCRIPTION OF EMBODIMENTS OF THE INVENTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety. Throughout the specification and claims, a given chemical formula or name shall encompass all optical and stereoisomers, as well as racemic mixtures where such isomers and mixtures exist.

Compositions for the delivery of active agents to immune cells are provided. The compositions comprise shape-specific micro and/or nanoparticles (i.e., having a predetermined geometry) with a broadest dimension less than about 10 μm having a pharmaceutically active component and an immune cell targeting component. The particles may additionally contain a non-pharmaceutically active component. The nanoparticles of the invention provide independent control over variables such as size, shape, composition, cargo encapsulation, surface functionality, and biodistribution to cells of the immune system. The micro and/or nanoparticles of the invention are formed of pharmaceutically active components and an immune cell targeting component. Additionally, other ingredients may be utilized in the micro and/or nanoparticles of the invention.

By “pharmaceutically active component” is intended a therapeutic or diagnostic active agent that is used in the formation of micro and/or nanoparticles. The micro and/or nanoparticles formed using an active agent and an immune cell targeting component can be used as therapeutic or diagnostic nanoparticles specific for cells of the immune system without the need for additional components. However, it is recognized that one or more non-pharmaceutically active components may be included with the active agent and the immune cell targeting component to form the micro and/or nanoparticles.

By “active agent” is intended an agent for delivery to the immune cells of a subject in need thereof. The active agent may find use in the treatment, diagnosis and/or management of a disease state. Such agents include but are not limited to small molecule pharmaceuticals, therapeutic and diagnostic proteins, immunogenic components, antibodies, DNA and RNA sequences, imaging agents, and other active pharmaceutical ingredients. Exemplary active agents include, without limitation, analgesics, anti-inflammatory agents (including NSAIDs), anticancer agents, antimetabolites, antineoplastic agents, immunosuppressants, antiviral agents, astringents, beta-adrenoceptor blocking agents, blood products and substitutes, contrast media, corticosteroids, diagnostic agents, diagnostic imaging agents, haemostatics, immunological agents, therapeutic proteins, enzymes, lipid regulating agents, prostaglandins, radio-pharmaceuticals, sex hormones (including steroids), anti-allergic agents, stimulants and anoretics, sympathomimetics, xanthines, antibiotics, and antiviral agents.

Anticancer agents include, without limitation, alkylating agents, antimetabolites, natural products, hormones, topoisomerase I inhibitors, topoisomerase II inhibitors, RNA/DNA antimetabolites, DNA antimetabolites, antimitotic agents and antagonists, and miscellaneous agents, such as radiosensitizers. Examples of alkylating agents include, without limitation, alkylating agents having the bis-(2-chloroethyl)-amine group such as chlormethine, chlorambucile, melphalan, uramustine, mannomustine, extramustinephoshate, mechlore-thaminoxide, cyclophosphamide, ifosfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carnustine, lomustine, semustine, or streptozotocine; and alkylating agents of the mitobronitole, dacarbazine and procarbazine type. See, for example U.S. Pat. No. 5,399,363. Antimitotic agents include allocolchicine, halichondrin B, colchicine, dolastatin, maytansine, rhizoxin, taxol and taxol derivatives, paclitaxel, vinblastine sulfate, vincristine sulfate, and the like. Topoisomerase I inhibitors include camptothecin, aminocamptothecin, camptothecin derivatives, morpholinodoxorubicin, and the like. Topoisomerase II inhibitors include doxorubicin, amonafide, m-AMSA, anthrapyrazole, pyrazoloacridine, daunorubicin, deoxydoxorubicin, mitoxantrone, menogaril, N,N-dibenzyl daunomycin, oxanthrazole, rubidazone, and the like. Other anticancer agents can include immunosuppressive drugs, such as cyclosporine, azathioprine, sulfasalazine, methoxsalen, and thalidomide.

Antimetabolites include, without limitation, folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine. Antibiotics also include gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefdinir, cefepime, teicoplanin, vancomycin, azithromycin, clarithromycin, cirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, bacitracin, colistin, polymyxin B, ciprofloxacin, enoxacin, gatifloxacin, levofloxacin, lomeflxacin, moxifloxacin, norfloxacin, ofloxacin, trovafloxacin, mafenide, prontosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincomycin, ethambutol, fusfomycin, fusidic acid, furazolidone, isoniazid, linezoilid, metronidazole, mupirocin, nitrofurantoin, platensimycin, pyrazinamide, quinupristin, dalfopristin, rifampin, rifampicin, tinidazole, etc.

Therapeutic proteins include enzymes, amylase, lipase, protease, blood factors, blood clotting factors, insulin, erythropoietin, interferons, including interferon-α and interferon-β transferrin, protein C, hirudin, granulocyte-macrophage colony-stimulating factor, somatropin, epidermal growth factor, albumin, hemoglobin, lactoferrin, angiotensin-converting enzyme, glucocerebrosidase, human growth hormone, VEGF, antibodies, monoclonal antibodies (including abciximab, adalimumab, alemtuzumab, basiliximab, bevacizumab, cetuximab, daclizumab, eculizumab, efalizumab, ibritumomab tiuxetan, infliximab, muromonab-CD3, natalizumab, omalizumab, palivizumab, panitumumab, ranibizumab, rituximab, traztuzumab, and the like), proteins naturally produced by the human body, recombinant versions of such proteins, and derivatives and analogs of such proteins, and soluble portions of receptors. For example, cytokines such as interleukins and interferons, and growth factors and soluble fractions of cytokine and growth factor receptors may be used. Proteins also include immunogenic components (e.g., those derived from an infectious organism), such as antigenic proteins or peptides and vaccines. Exemplary antigenic proteins/peptides include, without limitation, tetanus toxoid, typhoid toxoid, cholera toxoid, influenza HA antigen, influenza NP antigen, a human immunodeficiency virus (HIV) antigen, a Hepatitis B antigen, a Hepatitis C antigen, diphtheria toxoid, a Human papilloma virus (HPV) antigen, a feline leukemia virus (FeLV) antigen, a parvovirus antigen, and a distemper antigen. The Immune Epitope Database lists a number of defined epitopes that can be used as a suitable source for the immunogenic component of the presently disclosed subject matter. This database can be accessed on the World Wide Web at www.immuneepitope.org.

A subset of immunogenic components are specifically designed for cancer immunotherapy. The ex vivo delivery of purified tumor antigen to a defined population of immune cells (e.g., antigen presenting cells) may result in an effective tumor vaccine. Possible cancer immunotherapeutics which can be loaded into or on the targeted micro and/or nanoparticles include activated oncogene products (e.g., a mutated p21ras peptide, p210 product of bcr/abl rearrangement and HER-2/neu), tumor suppressor gene products (e.g., mutant p53 peptide), reactivated embryonic gene products (e.g., MAGE-1, MAGE-3, BAGE, GAGE, and RAGE), tissue specific differentiation antigens (e.g., MART-1, gp100, tyrosinase, a prostate specific membrane antigen, a prostate-specific antigen, and prostatic alkaline phosphatase), widely-expressed self proteins (e.g., carcinoembryonic antigen (CEA) and MUC-1), viral gene products (e.g., HPV^{b} E6 and E7 proteins, and Epstein-Barr virus EBNA-1^{c} proteins), idiotypic epitopes (e.g., immunoglobulin idiotypes and T cell receptor idiotypes), mesothelin, an active epitope of mesothelin, interleukin-13 receptor alpha 2, an active epitope of interleukin-13 receptor alpha 2, a breast cancer antigen, and an ovarian cancer antigen.

Essentially, any therapeutic protein desired to be delivered to an immune cell can be used in the formation of the micro and/or nanoparticles as the pharmaceutically active component. In some embodiments, the protein is capable of being dissolved, dispersed or otherwise being put into a solution, or withstanding elevated temperatures.

Optionally, the micro and/or nanoparticles having a pharmaceutically active component and an immune cell targeting component can further include a carrier. By “carrier” is intended a protein or polypeptide that has little or no therapeutic activity but is useful as a transport vehicle for an active agent. Such carrier proteins include albumin, modified albumin, hemoglobin, growth factor binding proteins, calcium binding proteins, acyl carrier proteins, and the like.

As indicated, the active agent may include a polynucleotide. The polynucleotide may be provided as an antisense agent or interfering RNA molecule such as an RNAi or siRNA molecule to disrupt or inhibit expression of an encoded protein. siRNA includes small pieces of double-stranded RNA molecules that bind to and neutralize specific messenger RNA (mRNA) and prevent the cell from translating that particular message into a protein. Alternatively, the polynucleotide may comprise a sequence encoding a peptide or protein of interest such as a therapeutic protein or antigenic protein or peptide. Accordingly, the polynucleotide may be any nucleic acid including but not limited to RNA and DNA. The polynucleotides may be of any size or sequence and may be single- or double-stranded. Methods for synthesis of RNA or DNA sequences are known in the art. See, for example, Ausubel et al. (1999) Current Protocols in Molecular Biology (John Wiley & Sons, Inc., NY); Sambrook et al. (1989) Molecular Cloning: A Laboratory Manual (2nd ed.) (Cold Spring Harbor Laboratory Press, Plainview, N.Y.).

Examples of natural products include vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifiers, such as alpha-interferon; camptothecin; taxol; and retinoids, such as retinoic acid.

Specific delivery of the active agent-containing micro and/or nanoparticles to immune cells is accomplished by the immune cell targeting component of the particles. By “immune cell targeting component” is intended a molecule that allows for the specific targeting of the active agent-containing micro and/or nanoparticles to immune cells. Exemplary immune cell targeting components include, without limitation, polypeptides, proteins, single-stranded nucleic acids, double-stranded nucleic acids, and small molecules. The immune cell targeting component can be a ligand for a cell surface receptor found on an immune cell, such as, for example, a CD11c ligand, a CD11b ligand, a CD11a ligand, a CD3 ligand, a CD4 ligand, a CD8 ligand, a CD18 ligand, a CD19 ligand, a CD20 ligand, a CD40 ligand, a CD205 ligand, a CMKLR1 ligand, a CD209 ligand, a CD83 ligand, a CD80 ligand, a CD86 ligand, a CCR7 ligand, a CD273 ligand, a DEC-205 ligand, or an Fc receptor ligand. The immune cell targeting component can also be an agonist for a cell surface receptor found on an immune cell, such as, for example, an Fc receptor agonist or a Toll-like receptor (TLR) agonist. TLR agonists include tri-acyl lipopeptides, lipoteichoic acid, double-stranded RNA, lipopolysaccharides, flagellin, diacyl lipopeptides, imidazoquinolines, and CpG-containing nucleotide sequences.

In some embodiments, the immune cell targeting component is an antibody (e.g., a monoclonal antibody) or antibody fragment (e.g., an Fab, an Fv, or a single-chain Fv). Such antibodies or antibody fragments will specifically bind cell surface proteins (e.g., receptors) found on immune cells, such as, for example, IgM, IgD, CD11c, CD11b, CD11a, CD3, CD4, CD8, CD18, CD19, CD20, and CD40. Cell surface proteins found on immune cells are well known in the art. See, for example, Janeway et al. (2005) Immunobiology, 6^{th} Ed. (Garland Science Publishing, New York, N.Y.). Likewise, antibody fragments and methods for making the same are well known in the art. See, for example, Harlow and Lane (1999) Using Antibodies: A Laboratory Manual, (Cold Spring Harbor Laboratory Press, Plainview, N.Y.).

The attachment of such immune cell targeting components would depend upon the targeting component and sites of attachment chosen that do no disrupt secondary structure. Protocols for linking using reactive groups and molecules are well known to one of skill in the art. See, e.g., Goldman et al. (1997) Cancer Res. 57: 1447-1451; Cheng (1996) Hum. Gene Therapy 7: 275-282; Neri et al. (1997) Nat. Biotechnol. 19: 958-961; Nabel (1997) Current Protocols in Human Genetics, vol. on CD-ROM (John Wiley & Sons, New York); Park et al. (1997) Adv. Pharmacol. 40: 399-435; Pasqualini et al. (1997) Nat. Biotechnol. 15: 542-546; Bauminger & Wilchek (1980) Meth. Enzymol. 70: 151-159; U.S. Pat. Nos. 6,280,760 and 6,071,890; and European Patent Nos. 0 439 095 and 0 712 621.

In some embodiments, targeting of the active agent-containing micro and/or nanoparticles to an immune cell (e.g., an APC, a B cell or a T cell) results in activation and/or proliferation of the targeted immune cell. In other embodiments, targeting of the active agent-containing micro and/or nanoparticles to an immune cell (e.g., an APC, a B cell or a T cell) results in stimulation of cytokine production in the targeted immune cell. In further embodiments, targeting of the active agent-containing micro and/or nanoparticles to an immune cell (e.g., an APC, a B cell or a T cell) results in apoptosis and/or death of the targeted immune cell.

By “immune cells” is intended any cell of the innate or adaptive immune system. Cells of the innate immune system include, without limitation, phagocytes (e.g., neutrophils, monocytes and macrophages), Natural Killer (NK) cells (also known as “large granular lymphocytes” (LGLs)), mast cells, basophils, eosinophils, and dendritic cells (DCs), which include interstitial DCs, Langerhans' cells of the skin and interdigitating cells of the thymus. Dendritic cells form an important bridge between innate and adaptive immunity, as these cells are capable of presenting antigenic peptides to T cells (as are macrophages and B cells; collectively these cells can be described as APCs). Dendritic cells lack surface expression of the differentiation antigens found on B cells (CD19 and CD20), T cells (CD3), monocytes (CD14), and natural killer cells (CD56), but they abundantly express molecules used for their specialized interactions with T cells. These include the antigen-presentation molecules CD1 and the class I and class II MHC proteins, the co-stimulatory molecules CD40, CD80/B7.1 and CD86/B7.2, and the adhesion molecules CD11a/LFA-1a, CD11b, CD11c, CD50/ICAM-3, CD54/ICAM-1, and CD58/LFA-3. While no DC-specific surface marker has been identified, the expression of CD83, a member of the immunoglobulin superfamily with unknown function, is relatively restricted to DCs. Dendritic cells are also known as professional APCs. Cells of the adaptive immune system include, without limitation, B cells and T cells (e.g., CD4+ T helper (T_{H}) cells, such as T_{H}1 and T_{H}2 cells, and CD8+ cytotoxic T cells).

As described herein, the micro and/or nanoparticles having a pharmaceutically active component and an immune cell targeting component may additionally contain a non-pharmaceutically active component. By “non-pharmaceutically active component” is intended a component that lacks pharmaceutical activity. The pharmaceutically active component and non-pharmaceutically active component may be bound to one another (covalently or non-covalently) or admixed with one another. In some embodiments, the non-pharmaceutically active component is a biocompatible material or polymer. Several biocompatible materials may be employed as the non-pharmaceutically active component of micro and/or nanoparticles of the invention. For example, in some embodiments, the particles are PEG hydrogel nanoparticles, composed of a mixture of PEG₄₂₈ triacrylate and PEG-carbonylimidazole-methacrylate in varying ratios. Other monomer combinations include PEG₄₂₈ triacrylate, PEG-monomethacrylate and a water soluble disulfide monomer in varying ratios. Still other monomer combinations include PEG₂₀₀-carbonylimidazole-methacrylate and trimethylolpropane ethoxylate triacrylate in varying ratios. Yet other monomer combinations include trimethylolpropane ethoxylate triacrylate, N,N-cystaminebisacrylamide and 2-aminoethylmethacrylate in varying ratios.

In other embodiments, synthetic biocompatible polymers can be included in the micro and/or nanoparticles of the invention. According to such embodiments, some examples include, but are not limited to, synthetic polypeptides containing one or more cross-linkable cysteine residues, synthetic polypeptides containing one or more disulfide groups, linear or branched chain polyalkylene glycols, polyvinyl alcohol, polyacrylates, polyhydroxyethyl methacrylate, polyacrylic acid, polyethyloxazoline, polyacrylamides, polyisopropyl acrylamides, polyvinyl pyrrolidinone, polylactide/glycolide, combinations thereof, and the like. According to further embodiments, synthetic polymers useful in combination with the particles of the invention include, but are not limited to, synthetic polyamino acids containing cysteine residues, synthetic polyamino acids containing disulfide groups, polyvinyl alcohol modified to contain free sulfhydryl groups, polyvinyl alcohol modified to contain free disulfide groups, polyhydroxyethyl methacrylate modified to contain free sulfhydryl groups, polyhydroxyethyl methacrylate modified to contain free disulfide groups, polyacrylic acid modified to contain free sulfhydryl groups, polyacrylic acid modified to contain free disulfide groups, polyethyloxazoline modified to contain free sulfhydryl groups, polyethyloxazoline modified to contain free disulfide groups, polyacrylamide modified to contain free sulfhydryl groups, polyacrylamide modified to contain free disulfide groups, polyvinyl pyrrolidinone modified to contain free sulfhydryl groups, polyvinyl pyrrolidinone modified to contain free disulfide groups, polyalkylene glycols modified to contain free sulfhydryl groups, polyalkylene glycols modified to contain free disulfide groups, combinations thereof, and the like. The biocompatible material/polymer can be biodegradable.

In some embodiments, the micro and/or nanoparticles of the invention are formed by a biocompatible material/polymer, and the pharmaceutically active component and/or the targeting component are attached to the biocompatible material/polymer. In other embodiments, the non-pharmaceutically active component (e.g., a biocompatible polymer) and the pharmaceutically active component are interspersed in the particle. In either instance, the pharmaceutically active component can be released from the particle via diffusion controlled release of the pharmaceutically active component (e.g., by varying the physical properties of the particle matrix, such as charge and mesh size), swelling of the particle matrix, chemical degradation of the non-pharmaceutically active component and/or the linkage between the pharmaceutically active component and the non-pharmaceutically active component, and enzymatic degradation of the non-pharmaceutically active component and/or the linkage between the pharmaceutically active component and the non-pharmaceutically active component. In further embodiments, release of the pharmaceutically active component is triggered by a change in physiological conditions following endocytosis or pinocytosis of the particle into an immune cell (e.g., an APC, a B cell or a T cell). In still further embodiments, the pharmaceutically active component is present in one or more pores of the non-pharmaceutically active component (e.g., a biocompatible polymer).

In some embodiments, the pharmaceutically active component can be combined with the biocompatible material/polymer components described herein to form a particle precursor material. According to such embodiments, the particle precursor material is then introduced to cavities of molds and formed into micro- or nanoparticles disclosed herein. According to some embodiments, the particle precursor material can include the biocompatible material/polymer component without the pharmaceutically active component. According to embodiments where the particle precursor material does not include the pharmaceutically active component, the active agent can be introduced into and/or onto the particle after the particle is fabricated.

The predetermined geometries of the immune cell-targeted micro and/or nanoparticles of the invention include substantially spherical, substantially non-spherical, substantially viral shaped, substantially bacteria shaped, substantially protein shaped, substantially cell shaped, substantially rod shaped (e.g., where the rod is less than about 200 nm in diameter), substantially chiral shaped, substantially a right triangle, substantially flat disc shaped with a thickness of about 2 nm, substantially flat disc shaped with a thickness of greater than about 2 nm, and substantially boomerang shaped. In one embodiment, the particles have a broadest dimension less that about 1 micron, for example, between about 1 nm and about 500 nm (such as between about 50 nm and about 200 nm).

The immune cell-targeted micro and/or nanoparticles of the invention are fabricated using PRINT™ technology (Particle Replication in Non-wetting Templates) (Liquidia Technologies, Inc., Research Triangle Park, N.C.), which allows the combination of effective targeting to immune cells (e.g., APCs, B cells and T cells) with the ability to carry active agents (e.g., anticancer agents and immunogenic components, such as antigens) unaltered by extracellular process into those immune cells. PRINT™ technology utilizes photochemically curable perfluoro-polyether-based elastomers (PFPEs) to replicate micro or nano sized structures on a master template. The polymers utilized in PRINT™ molds are liquid at room temperature and can be photo-chemically cross-linked into elastomeric solids that enable high resolution replication of micro or nano sized structures. The liquid polymer is then cured while in contact with the master, thereby forming a replica image of the structures on the master. Upon removal of the cured liquid polymer from the master template, the cured liquid polymer forms a patterned template that includes cavities or recess replicas of the micro or nano-sized features of the master template and the micro or nano-sized cavities in the cured liquid polymer can be used for high-resolution micro or nanoparticle fabrication. PRINT™ technology enables the fabrication of monodisperse organic nanoparticles with simultaneous control over structure (i.e. shape, size and composition) and function (i.e. cargo and surface structure).

PRINT™ technology is the first general, singular method capable of forming organic particles that:

i) are monodisperse in size and uniform shape;

ii) can be molded into any shape;

iii) can be comprised of essentially any matrix material;

iv) can be formed under extremely mild conditions (compatible with delicate cargos);

v) is amenable to post functionalization chemistry (e.g., bioconjugation of active agents and/or targeting components); and

vi) which initially fabricates particles in an addressable 2-D array (which opens up combinatorial approaches since the particles can be “bar-coded”).

Technical aspects to be considered when designing a PRINT™ nanoparticle carrier system include: 1) compatibility of the cargo with the prepolymer PRINT™ matrix, 2) particle degradation profile desired for cargo release, 3) targeting method, 4) particle modulus, and 5) the combination of points 1-4 in the formation of a prepolymer mixture that is amenable to the PRINT™ process outlined herein. Cargo/prepolymer matrix compatibility can be addressed by tuning the hydrophilicity of the prepolymer matrix to match that of the cargo through judicious choice of monomers. Particle degradation and targeting are discussed herein. Modulus can be adjusted by changing the degree of crosslinking within the particle. Finally, the particle formulation can be optimized for PRINT™ fabrication, if needed, by adding co-monomers or co-solvents to alter the physical properties of the monomer solution.

For more detailed description of the materials used to fabricate the molds of the present invention and methods of molding micro or nanoparticles in the molds see U.S. patent application Ser. Nos. 10/583,570, filed Jun. 19, 2006, and 11/594,023 filed Nov. 7, 2006; and PCT International Patent Application Serial Nos.: PCT/US04/42706, filed Dec. 20, 2004; PCT/US/06/23722, filed Jun. 19, 2006; PCT/US06/34997, filed Sep. 7, 2006; PCT/US06/43305, filed Nov. 7, 2006; and PCT/US07/02476, filed Jan. 29, 2007; each of which is incorporated herein by reference in its entirety. See also, U.S. Provisional Patent Application Ser. Nos. 60/531,531, filed Dec. 19, 2003; 60/583,170, filed Jun. 25, 2004; 60/604,970 filed Aug. 27, 2004; 60/691,607, filed on Jun. 17, 2005; 60/714,961, filed Sep. 7, 2005; 60/762,802, filed Jan. 27, 2006; 60/798,858, filed May 9, 2006; 60/734,228, filed Nov. 7, 2005; 60/757,411, filed Jan. 9, 2006; 60/799,876, filed May 12, 2006; 60/833,736, filed Jul. 27, 2006; and 60/828,719, filed Oct. 9, 2006; each of which is incorporated herein by reference it its entirety.

The micro and/or nanoparticles formed using an active agent and an immune cell targeting component of the invention are formulated such that a therapeutically effective amount of a plurality of monodisperse particles can be administered to a subject in need thereof. The term “therapeutically effective amount” as used herein refers to an amount of the plurality of monodisperse particles sufficient to achieve a certain outcome, such as to target an immune cell of the subject or elicit an immune response in the subject. In reference to targeting an immune cell to inhibit or prevent unwanted cell proliferation, a therapeutically effective amount comprises an amount sufficient to prevent or delay unwanted cell proliferation. By “eliciting an immune response” is intended the generation of a specific immune response (or immunogenic response) in a vertebrate. In some embodiments, the immunogenic response is protective or provides protective immunity, in that it enables the vertebrate animal to better resist infection or disease progression from the organism or tumor cell against which the immunogenic composition is directed.

The immune cell-targeted micro and/or nanoparticles described herein may be present in a dry formulation or suspended in a biocompatible medium to prepare a pharmaceutical composition. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The pharmaceutical composition of the invention can include other agents, excipients or stabilizers. For example, to increase stability or decrease non-specific uptake by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-.alpha.-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, for example, sodium cholesteryl sulfate and the like. Similarly, the positive zeta potential of nanoparticles can be altered by adding positively charged components.

Formulations suitable for oral administration can consist of (a) liquid solutions, such as an effective amount of the particles dissolved in diluents, such as water, saline, or orange juice, (b) capsules, sachets or tablets, each containing a predetermined amount of the particles, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the particles in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable pharmaceutical carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Pharmaceutical formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the pharmaceutical composition is formulated to have a pH range of about 4.5 to about 9.0, including for example pH ranges of any of about 5.0 to about 8.0, about 6.5 to about 7.5, and about 6.5 to about 7.0. In some embodiments, the pH of the pharmaceutical composition is formulated to no less than about 6, including, for example, no less than about any of 6.5, 7 or 8 (such as about 8). The pharmaceutical composition can also be made to be isotonic with blood by the addition of a suitable tonicity modifier, such as glycerol.

The pharmaceutical compositions comprising the immune cell-targeted micro and/or nanoparticles described herein can be administered to a subject (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the nanoparticle composition can be administered by inhalation to target immune cells of the respiratory tract. In some embodiments, the nanoparticle composition is administrated intravenously. In some embodiments, the nanoparticle composition is administered orally.

The pharmaceutical compositions of the invention provide for better biodistribution of the immune cell-targeted micro and/or nanoparticles upon administration, and additionally allow for enhanced stability. In this manner, more of the active agent is delivered at the target site.

The following examples are offered by way of illustration and not by way of limitation.

### EXAMPLES

### Example 1

### Preparation of Peg Hydrogel Particles for Dendritic Cell Targeting Studies

An 8 inch silicon substrate patterned with 200 nm tall×200 nm diameter cylindrical shapes is placed under a UV source. A patterned perfluoropolyether (PFPE) mold is generated by pouring 20 mL of PFPE-dimethacrylate (PFPE-DMA) containing 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

The following polymer composition is used:

Particle Composition:

| Monomer | wt % |
|---|---|
| PEG₄₂₈ triacrylate | 59 |
| PEG-carbonylimidazole-methacrylate | 40 |
| 2,2-diethoxyacetophenone | 1 |
| Total | 100 |

PEG₄₂₈ triacrylate (590 mg), PEG-carbonylimidazole-methacrylate (400 mg), and 2,2-diethylacetophenone (1 mg) were combined in an Eppendorf tube and then mixed for approximately 30 seconds on a vortexer. Approximately 120 μL, of this solution was spotted onto the 8 inch PFPE mold generated above. A polyethylene (PE) sheet was then placed atop the spotted monomer solution on the mold. A small roller was then used to apply force over the PE sheet to facilitate spreading of the monomer solution to coat the entire mold. The PE sheet was removed by slowing peeling the sheet away from the mold. After peeling, the filled mold was placed under a UV curing source, purged with nitrogen for two minutes to remove oxygen, and finally subjected to UV light (λ=365) for two minutes. The fully cured particles were then harvested by placing DMSO on the mold and scraping the surface with a glass slide (4 times using 0.5 mL DMSO each time). The particle-containing DMSO solution was collected in a glass scintillation vial.

### Example 2

### Procedure for Surface Functionalization

Five molds of particles prepared using the procedure described above were harvested into 10 mL DMSO. Streptavidin (Alexa Fluor 647) was conjugated to the surface by adding 400 μL of a 2 mg/mL solution in PBS buffer (Invitrogen) to the particle solution and stirring for 5 hours at room temperature while excluding light. The solutions were then placed in a refrigerator and stored for 72 hours at 4° C. Water (20 mL) was added and the solution was filtered (Fisherbrand P8, 20-25 μm pore size) by vacuum filtration. The particles were collected onto three centrifugal filter membranes (0.1 μm pore size, PVDF membrane, Millipore) and then washed with 16 mL of water to remove any unbound streptavidin. The particles were resuspended in approximately 4 mL of water.

### Example 3

### Procedure for Surface Deactivation

2-Amino-1-ethanol (270 μL) was added to the 4 mL particle solution from above and the mixture was stirred for 1 hour (h) at room temperature. The particles were collected from solution onto a centrifugal filter membrane (0.1 μm pore size, PVDF membrane, Millipore) and then washed with 4 mL of water to remove any residual 2-amino-1-ethanol.

### Example 4

### Synthesis of the Water Soluble Disulfide

The development of a water soluble disulfide that can be used in the PRINT™ process is desirable. Issues related to miscibility with the monomer systems and mechanical stability of the particle must be addressed. To address these issues the diacrylate and the tetraacrylate version of the disulfide have been synthesized. See Scheme 1.

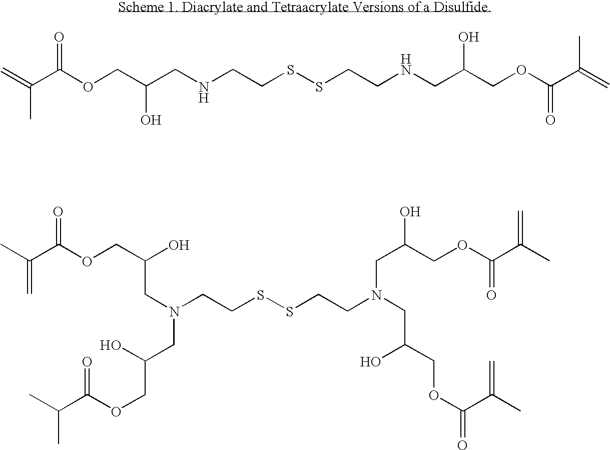

The following procedure was used: Glycidylmethacrylate (4 g, 28 mmol) was added to 20 mL acetonitrile followed by the addition of Mg(ClO₄) (3.0 g, 14 mmol). The mixture was stirred for 20 min in a water bath before adding cystamine (1.08 g, 7 mmol) dropwise (extremely exothermic reaction). After stirring for 3 days, 15 mL of water was added followed by extraction with chloroform (3×30 mL). The solution was then filtered through a plug of alumina, and the remaining solvent removed by rotovap leaving a yellow oil (3.3 g, 65% isolated yield).

This disulfide is miscible with PEG₆₀₄-triacrylate and forms a very strong material when polymerized as a 50:50 mix. 200 nm particles using the hydrophilic disulfide tetramethacrylate (HDT) monomer were fabricated with and without doxorubicin incorporated as a cargo.

The particles were harvested and filtered in chloroform. This strategy prevents aggregation of disulfide particles during purification. Furthermore, conjugation of streptavidin to the surface should sterically stabilize the particles, which should make it easy to disperse them in PBS.

Particle formulation 1: (PRINT™ particles fabricated as a 90:10 solids:DMSO): PEG₆₀₀-triacrylate—65 wt %, HDT—33 wt %, Doxorubicin—1 wt %,

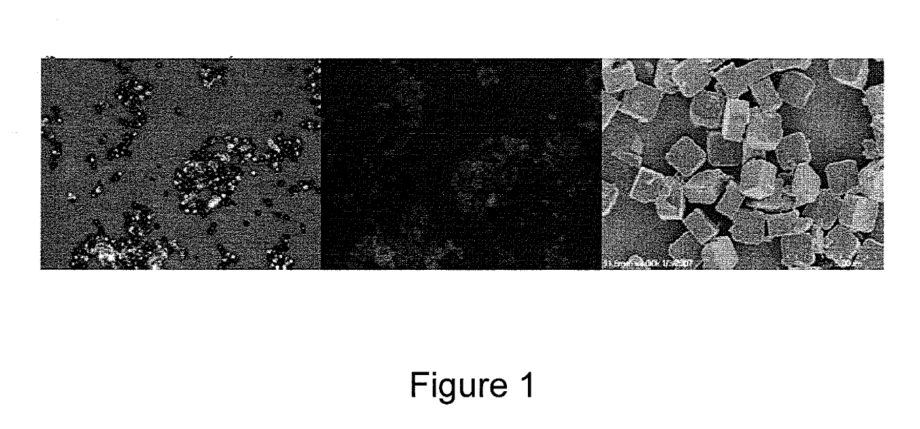

DEAP—1 wt %. These particles were harvested in DMSO and washed several times to remove free doxorubicin (see FIG. 1).

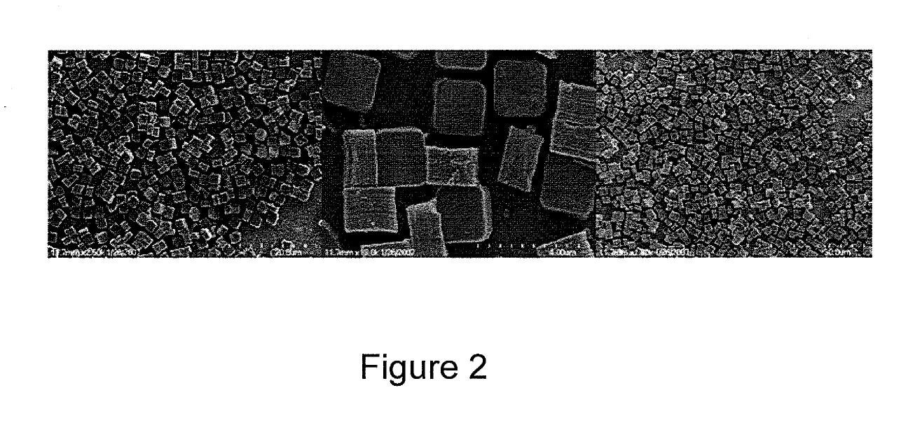

Particle formulation 2: (PRINT™ particles fabricated as a 80:20 solids:DMSO): PEG₆₀₀-triacrylate—66 wt %, HDT—33 wt %, DEAP—1 wt %. These particles were harvested in CHCl₃, reacted with CDI overnight, and then purified. Streptavidin Alexa Fluor 647 can be conjugated to the surface (see FIG. 2).

Other surface functionalization strategies will involve reacting the hydroxyl groups of the HDT monomer with carbonyldiimidazole post-PRINT™ fabrication to functionalize the surface of these particles.

### Example 5

### Optimization of Ligand Binding to Dendritic Cells

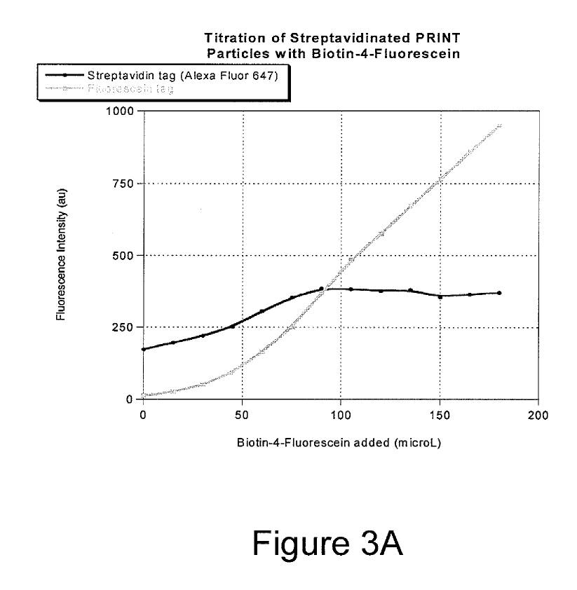

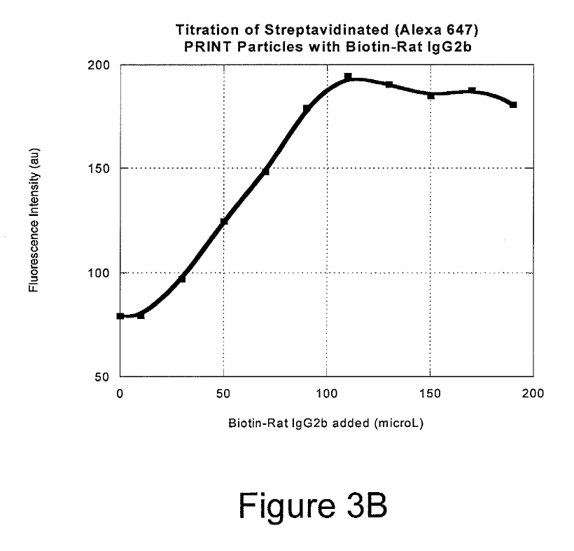

First was to alter the particle surface to decrease the non-specific binding. The initial PRINT™ particles used showed high levels of non-targeted uptake/binding by nearly all cells. Experiments to block the non-targeted binding ultimately revealed that unreacted functional groups on the PRINT™ particle surface resulted in covalent attachment to cell surfaces. This non-specific binding could be largely eliminated by treating the PRINT™ particles with ethanolamine following avidin coupling. The second was to determine the optimum concentration of the streptavidin coupling to the surface of the PRINT™ particle to allow control of the antibodies used for targeting. The concentration of streptavidin on the surface of PRINT™ particles was measured by titration with biotin-4-fluorescein. As biotin-4-fluorescein is titrated into a solution containing streptavidin it quickly binds altering the fluorescence intensity of the fluorescein tag. When binding is to free streptavidin in solution fluorescence is quenched; however, when the streptavidin is bound to PRINT™ particles more complicated behavior is observed. This phenomenon is not completely understood, but the response eventually becomes linear in both cases, which can be taken as an indication that all available binding sites have been filled. Changes are also observed in the fluorescence intensity of the Alexa Fluor 647 tag on streptavidin upon binding of biotin. Specifically, a PRINT™ particle solution was titrated with biotin-4-fluorescein while monitoring the fluorescence of both the fluorescein tag and the Alexa Fluor 647 tag on streptavidin (see FIG. 3A). The fluorescence of the fluorescein tag begins to increase linearly after the addition of approximately 90 μL of biotin. At the same time, the fluorescence of the Alexa Fluor 647 tag stabilizes. These two pieces of data indicate that all available binding sites have been filled after the addition of 90 μL of biotin. Assuming the mass of a single cylindrical PRINT™ particle (200 nm d×200 nm h) to be 3.35×10⁻¹⁵ g, there were ˜1240 biotin binding sites per particle. If it is further assumed that there are three available binding sites per copy of streptavidin (one binding site is disrupted by attachment to the particle) there were ˜412 copies of streptavidin per particle.

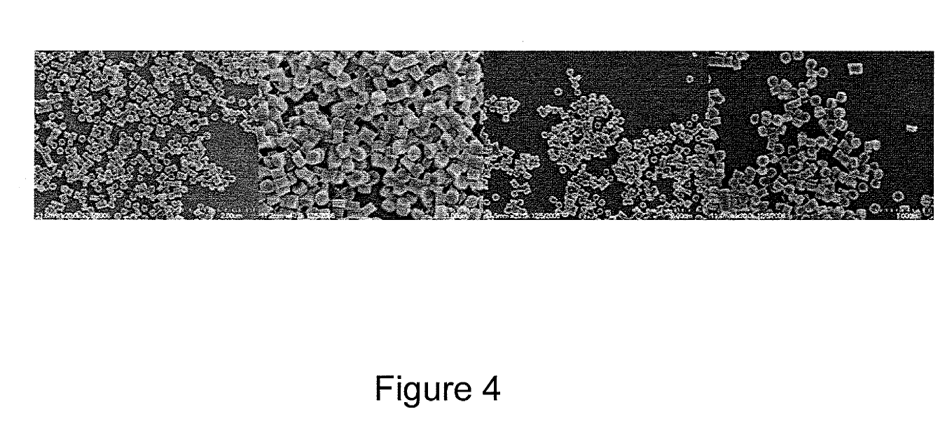

The amount of biotin rat IgG2b bound to the surface of PRINT™ particles was measured by titration of a streptavidinated PRINT™ particle solution with biotin rat IgG2b isotype control. This assay takes advantage of the knowledge gained from the previous experiment where the number of copies of streptavidin per PRINT™ particle was determined. It was discovered that biotin binding, in addition to affecting the fluorescence of biotin-4-fluorescein, affects the fluorescence intensity of the Alexa Fluor 647 tag on streptavidin. A PRINT™ particle solution was titrated with a stock antibody solution while monitoring the fluorescence of the Alexa Fluor 647 tag on streptavidin (see FIG. 3B). Again, behavior similar to that of the biotin-4-fluorescein titration was observed for the Alexa tag. The fluorescence signal increased in intensity as the biotinylated antibody was added initially, and at a certain point the readings began to stabilize as additional antibody was added. It was assumed that all accessible binding sites were filled when the readings began to stabilize. Based on a molecular weight of 150 kDa for the antibody, this corresponds to ˜380 copies of the antibody per particle. This number correlates well with the number of copies of streptavidin per particle (412) and is the expected result based on a one to one binding ratio. In addition to rat IgG2b, anti-mouse CD11b, anti-mouse CD11c, anti-mouse CD80, and Armenian hamster IgG isotype antibodies have routinely been added to PRINT™ particles (see FIG. 4).

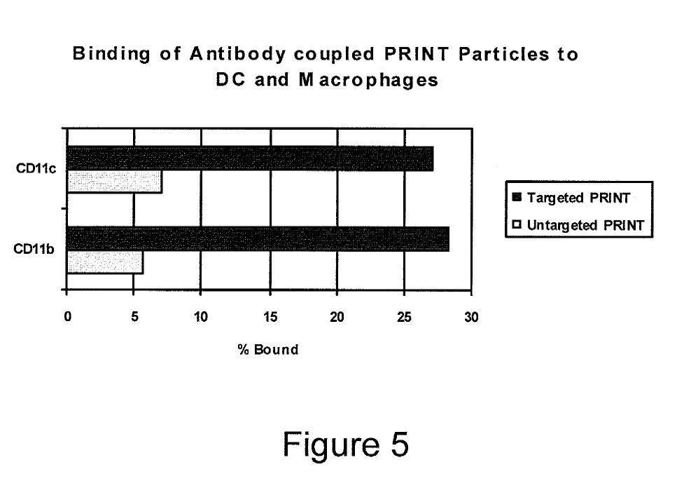

The initial concentration of coupled avidin was too low to allow effective binding of PRINT™ particles to professional APC. As a result the coupling ratios were altered to increase the number of avidin molecules/PRINT™ particle. This has allowed effective targeting. FIG. 5 shows the targeting efficacy, illustrating the increased specific binding. The sensitivity can likely be increased by higher coupling efficiencies.

### Example 6

### Stimulation of Immune Response by PRINT™ Particles

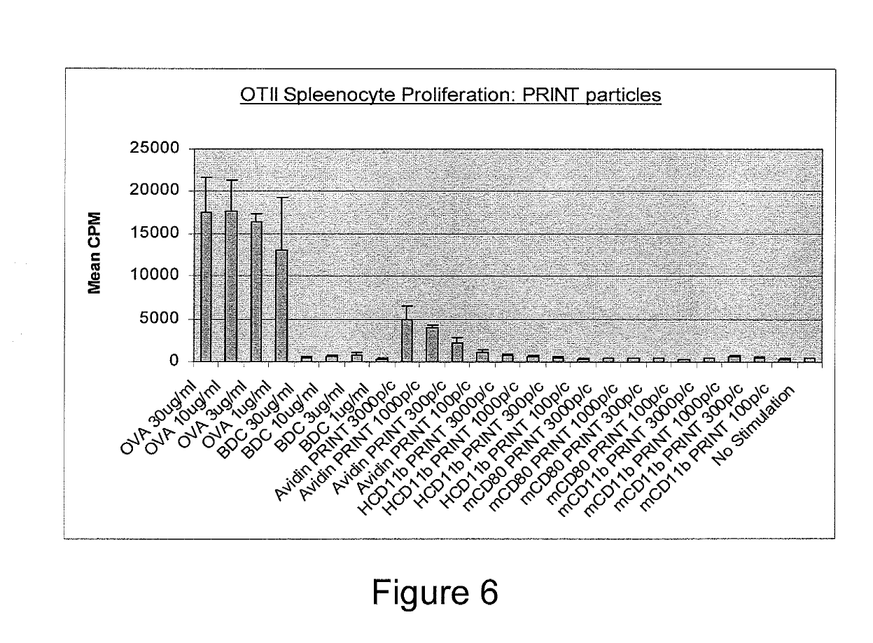

As shown in FIG. 6, the CD80-PRINT™ and CD11b-PRINT™ particles themselves did not stimulate any proliferative response. The PRINT™ particles that were not conjugated to antibody (e.g. the Avidin only PRINT™ particles) seem to stimulate spleenocyte proliferation. This is likely due to the nonspecific stickiness of avidin for receptors on the surface of the T cells, leading to crosslinking of unknown stimulatory receptors. When the antibodies to CD80 and CD11b were on the particles, they probably blocked access to the avidin. The strong response to the avidinated particles was a little surprising. The non-specific binding interactions were greatly attenuated in the antibody-coated particles.

### Example 7

### Assessment of Impact of PRINT™ Particles on Immune Response

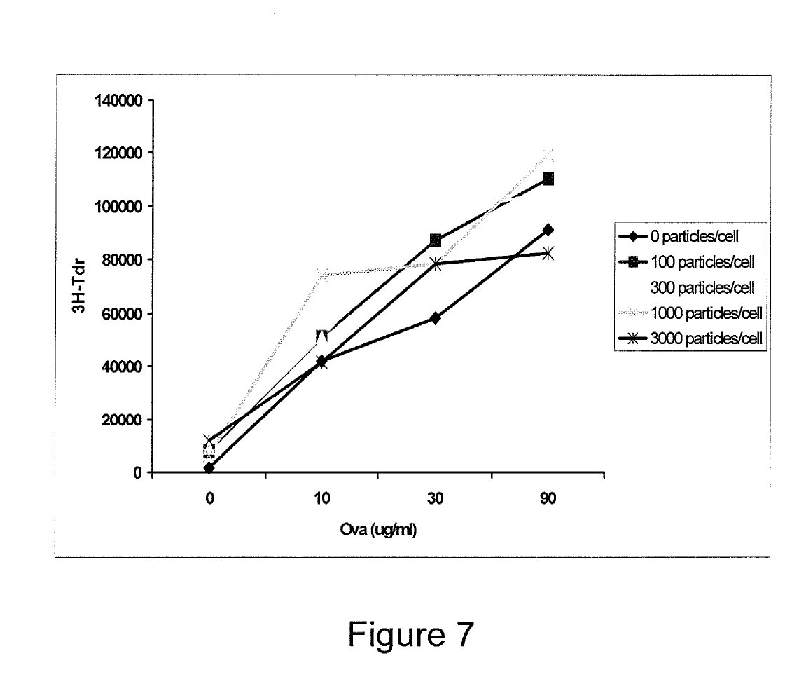

It was determined that the PRINT™ particles themselves have no nonspecific impact on immune responses in vitro. The immune response to ovalbumin was titrated in vitro. In this experiment, titrations of ova were performed in the presence of different levels of PRINT particles. As seen in FIG. 7, no evidence of inhibition of the ova response was seen, using proliferation of OT-II T cells as readout. Exposure to up to 3000 PRINT™ particles per cell has no effect on the ability of T cells to recognize ovalbumin. Ovalbumin loaded as a cargo in PRINT™ nanoparticles can be used to stimulate OT1 and OTII T cells in vitro.

### Example 8

### Conjugation of Targeting Ligands

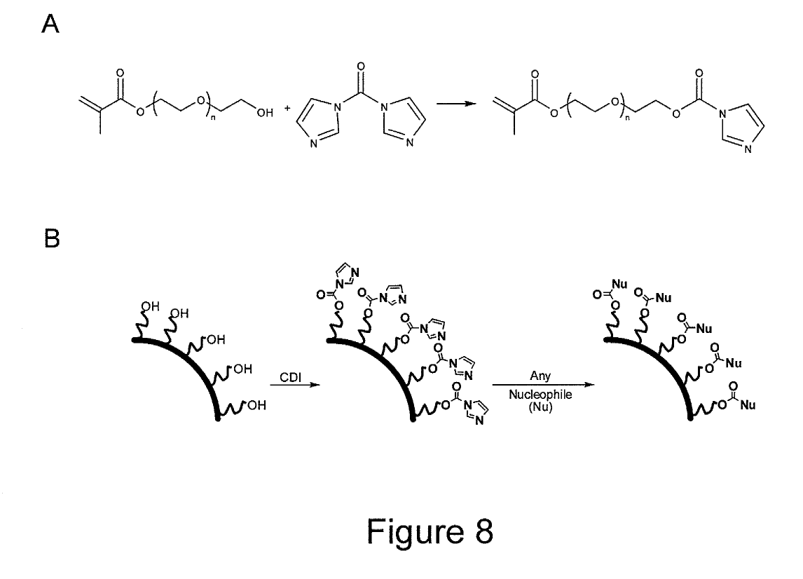

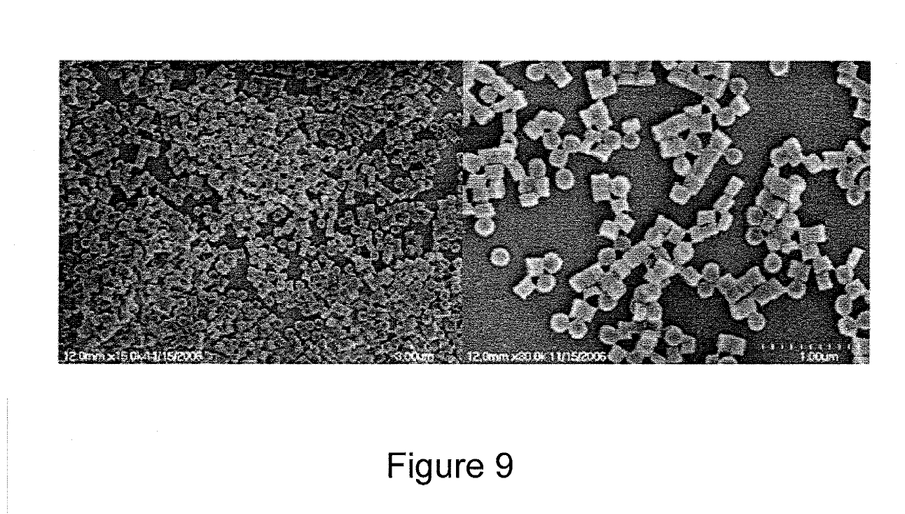

Effective conjugation chemistries allow us to conjugate a wide range of targeting ligands to the surface of the PRINT™ particles. A general surface coupling strategy was developed, which utilizes carbonyldiimidazole as the coupling reagent. The hydroxyl group of PEG₄₈₅-monomethylmethacrylate can be reacted either pre- or post-PRINT™ fabrication with carbonyldiimidazole to form a carbonylimidazole-modified PEG₄₈₅-monomethacrylate (see FIG. 8). Virtually any moiety containing a nucleophilic species can then be covalently attached to PRINT™ particles. Particles were synthesized using preformed carbonylimidazole monomer with the following composition: PEG₄₂₈-triacrylate (59 wt %), PEG₄₈₅-carbonylimidazole-monomethacrylate (40 wt %), and 2,2-diethoxyacetophenone (1 wt %). SEM images are shown in FIG. 9.

Initial studies have focused on conjugating streptavidin to the particle surface followed by the addition of a biotinylated targeting ligand. This method allows a number of targeting ligands to be screened from a large pool of commercially available biotinylated reagents.

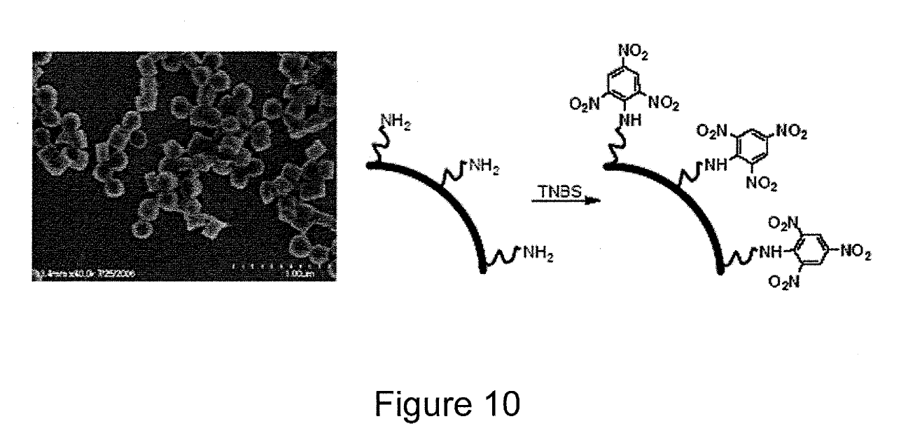

In order to effectively conjugate nitrophenyl onto the surface of PRINT™ particles, conjugation chemistries are needed so as to functionalize the PRINT™ particles with targeting ligands and small molecules. A second strategy for surface modifying PRINT™ particles has been developed based on installing reactive amine handles on the surface. Particles with as much as 11 wt % aminoethylmethacrylate (AEM) incorporated have been prepared (see FIG. 10) Amine groups on the surface will be reacted with 2,4,6-trinitrobenzene 1-sulfonic acid (TNBS) which will produce surface conjugated nitrophenyl groups (NP) (see FIG. 10). NP surface coverage can be varied trivially by changing the amount of AEM in the particle matrix. The use of TNBS also allows for quantitative spectrophotometric determination of the number of amine groups on the surface of each particle composition.

### Example 9

### Synthesis of Carbonyl-Imidazole-Modified Methacrylates

PEG₂₀₀-monomethacrylate (6.4 g) was dissolved in chloroform (75 mL) at room temperature (rt). Carbonyldiimidazole (5.1 g dissolved in 50 mL chloroform) was then added at rt and the flask was stirred for 1.5 hours. The solution was washed with de-ionized water (4×100 mL), dried with over magnesium sulfate, and the solvent removed by rotovap leaving 7.8 g of a clear slightly yellow oil (92% yield).

### Example 10

### T-Independent Response

A T-independent response requires only interaction with antigen specific B cells. To test the antibody responses PRINT™ particles decorated with pneumococcal polysaccharide-C (Pnp) will be used. Two experiments will be performed; first mice will be injected directly with the PRINT™ particles and serum antibody responses measured on days 4 and 21 using class specific ELISA assays on Pnp coated plates. Particular attention will be paid to IgM responses that would be expected in a T-independent response. In addition, IgG responses will also be measured in case the high ligand density allows T-independent class switching.

### Example 11

### PRINT Particles Having Pneumococcal Polysaccharide (Type 5) Conjugated to the Surface

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 60 mg PEG₂₀₀-CI-methacrylate, 39 mg trimethylolpropane ethoxylate triacrylate (˜428 MW), 1 mg 2,2-diethoxyacetophenone was prepared. This mixture was spotted directly onto the patterned PFPE-DMA mold and covered with a polyethylene (PE) film. The monomer mixture was pressed between the two polymer sheets, and spread using a roller. The PE sheet was then slowly peeled from the mold and then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial.

### Pnp Coating

Five molds of particles with the composition listed above were harvested into 10 mL DMSO. Pneumococcal polysaccharide (type 5) (American Type Culture Collection) was conjugated to the surface by adding 500 μL of a 10 mg/mL solution in water to the particle solution and stirring for overnight at rt. Water (10 mL) was added and the solution was filtered (Fisherbrand P8, 20-25 μm pore size) by vacuum filtration. The particles were collected onto two centrifugal filter membranes (0.1 μm pore size, PVDF membrane, Millipore) and then washed with 10 mL of water to remove any unbound polysaccharide. The particles were re-suspended in ˜8 mL of water.

### Surface Deactivation

2-Amino-1-ethanol (450 μL) was added to the 8 mL particle solution from above and the mixture was stirred for 0.5 h at rt. The particles were collected from solution onto a centrifugal filter membrane (0.1 μm pore size, PVDF membrane, Millipore) and then washed with 4 mL of water to remove any residual 2-amino-1-ethanol. The particles were re-suspended in 1.5 mL, and analyzed by TGA (2.0 mg/mL). PBS (150 μL, 10×) was added to 1350 μL of particle solution leaving 1.5 mL of a 1.8 mg/mL particle solution in PBS.

Pnp-coated particles had a size of 258 nm (as determined by DLS), a PDI of 0.124 and a zeta potential of −21.6 mV.

### Example 12

### Stimulate B Cell Response

To test the ability of PRINT™ particles to stimulate B cells directly, NP (nitrophenyl)-decorated PRINT™ particles will be added to purified NP specific splenic B cells derived from the B cell receptor transgenic mouse, B8-1. The transgenic mouse allows a greater sensitivity for response. Immunoglobulin production in those cultures will be assayed as a function of nanoparticle dose and ligand density. The expectation is that the antigen bearing PRINT™ particles will stimulate a direct TI-2 response resulting in IgM production directed at the antigen.

### Example 13

### Stimulate T Cell Responses

The ability to stimulate T cell responses by targeting particles to specific receptors on antigen presenting cells will be determined. To test the ability of PRINT™ particles to stimulate B cells directly, NP (nitrophenyl)-decorated PRINT™ particles will be added to purified NP specific splenic B cells derived from the B cell receptor transgenic mouse, B8-1. The transgenic mouse allows a greater sensitivity for response. Immunoglobulin production will be assayed in those cultures as a function of nano-particle dose and ligand density. The expectation is that the antigen bearing PRINT™ particles will stimulate a direct TI-2 response resulting in IgM production directed at the antigen, both in vivo and in vitro. In addition cargo can be incorporated to enhance the function of these nanoparticles. For example, using pH sensitive particles including CpG as cargo to simulate B cells through TLR9. For T cell responses the effect of PRINT™ particles in vivo and in vitro will again be assayed. For in vivo experiments the particles will be targeted to dendritic cells using particles decorated with an antibody specific for DEC 205 (CD205). This will allow the efficient uptake of the particles by dendritic cells via receptor meditated endocytosis. The PRINT™ particles will be loaded with ovalbumin. These particles will be injected intravenously. To detect T cell activation by dendritic cells in vivo proliferation of CD4+ and CD8+ transgenic T cells will be measured. B6 mice will be injected with CFSE labeled CD8+ OT-1 T cells and Cell Tracker Red labeled CD4+ OT2 T cells one day before the nanoparticles. Both cells respond to ovalbumin derived peptides processed by dendritic cells. Following nano-particle injection, proliferation will be measured by dye dilution using flow cytometry. To assess function in vitro, bone marrow derived dendritic cells will be treated with PRINT™ particles. For these experiments cargos such as quantum dots or some other fluorophore will be explored. Targeted and non-targeted particles for uptake by dendritic cells can then be compared. For a functional assay the PRINT™ particles with ovalbumin cargo will be used to stimulate OT1 and OTII T cells in vitro. Treated cells will be titrated to assess antigen presenting competence. Controls both in vitro and in vivo will include PRINT™ particles with a control cargo (hen egg lysozyme). The composition of the PRINT™ particles will also be alterable to be comprised of a hydrogel matrix material to allow them to dissolve in the endoplasmic reticulum using cross-linkers that are sensitive to the reducing environment of the endosomes or a pH sensitive linker that is sensitive to the lowered pH of endosomes. The expected result is that it will be possible to induce proliferation of both CD4+ and CD8+ T cells in vivo, as well in vitro.

### Example 14

### PRINT™ Nanoparticles Designed for Release of Ovalbumin In Vitro

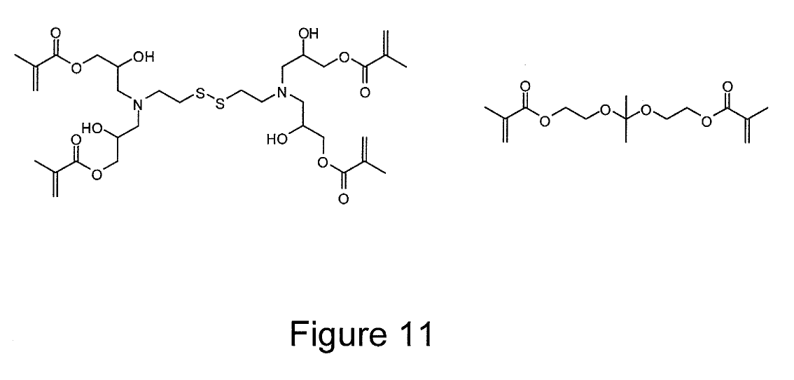

PRINT™ particles have been fabricated that are comprised of a hydrogel matrix material to allow them to dissolve in the endoplasmic reticulum using disulfide cross-linkers that are sensitive to the reducing environment (see FIG. 11). Other cross-linkers could be substituted that degrade in response to changes in pH, such as the acetal cross-linker shown in FIG. 11.

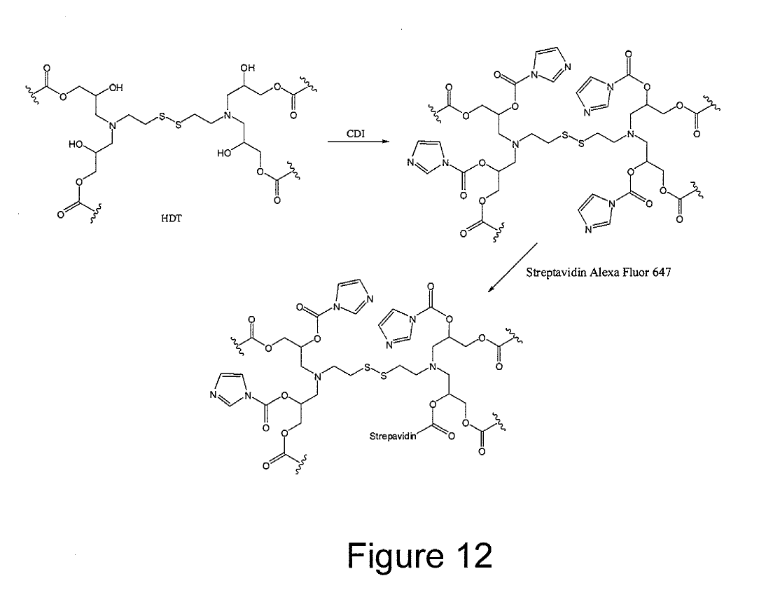

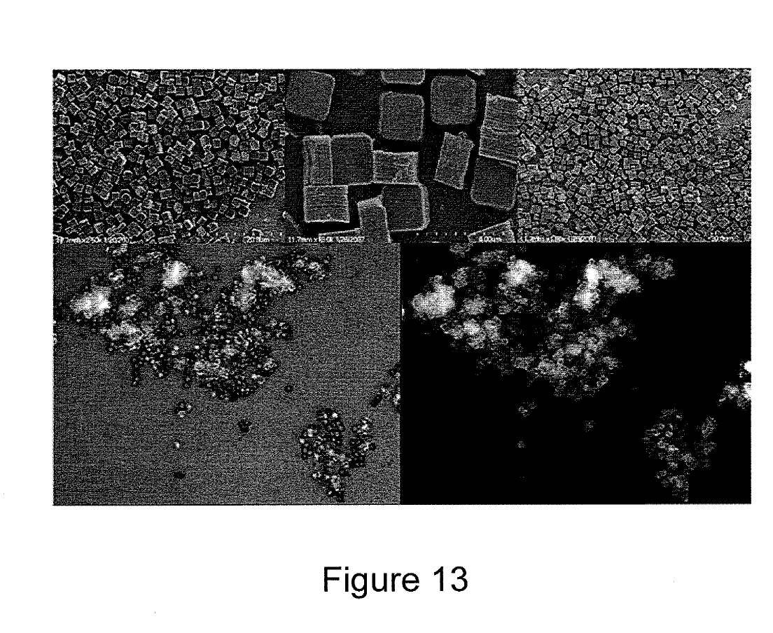

Streptavidin can be attached to the surface of these particles using the same strategy outlined previously for the attachment of avidin to 100% PEG-based particles. In this scenario, the four hydroxyl groups of the disulfide monomer are converted to reactive carbonylimidazole groups (see FIG. 12). These surface activated particles were then reacted with streptavidin Alexa Fluor 647. The particles were analyzed by SEM, optical, and fluorescence microscopy (see FIG. 13).

To assess function in vitro, bone marrow derived dendritic cells will be treated with PRINT™ particles. Targeted and non-targeted particles for uptake by dendritic cells can then be compared. For a functional assay the PRINT™ particles with ovalbumin cargo will be used to stimulate OT1 and OTII T cells in vitro.

### Example 15

### PRINT™ Nanoparticles Designed for Release of Ovalbumin In Vivo

PRINT™ nanoparticles will be fabricated for cargo release. For in vivo experiments the particles will be targeted to dendritic cells using particles decorated with an antibody specific for CD11c. This will allow the efficient uptake of the particles by dendritic cells via receptor meditated endocytosis. The PRINT™ particles will be loaded with ovalbumin. These particles will be injected intravenously. To detect T cell activation by dendritic cells in vivo proliferation of CD4+ and CD8+transgenic T cells will be measured. B6 mice will be injected with CFSE labeled CD8+ OT-1 T cells and Cell Tracker Red labeled CD4+ OT2 T cells one day before the nanoparticles. Both cells respond to ovalbumin derived peptides processed by dendritic cells. Following nanoparticle injection proliferation will be measured by dye dilution using flow cytometry.

### Example 16

### Synthesis of Disulfide-Based, Anti-CD11b Targeted, Ova Peptide-Containing PRINT Particles

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 2×2×1 μm cubes. The PFPE-DMA covered master was then subjected to UV light (λ=365 nm) for 3 minutes while under a nitrogen purge. The fully cured PFPE-DMA mold was then released from the silicon master. Separately, a mixture of 67 mg trimethylolpropane ethoxylate triacrylate (˜900 MW), 20 mg N,N-cystaminebisacrylamide, 10 mg 2-aminoethylmethacrylate, 2 mg ova peptide (SIINFEKL, Anaspec, Inc.), 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm²) for 2 minutes while maintaining a nitrogen purge. The particles were harvested by placing 2 mL of chloroform on the mold and scrapping the surface with a glass slide. The particle suspension was transferred to a scintillation vial.

### Biotinylation

One mold of particles with the composition listed above was harvested into 9 mL of water. PBS (1 mL, 10×) was added followed by Sulfo-NHS-LC-Biotin (100 μL, 45 mM in DMSO) and the mixture was stirred for 0.5 h. Particles were collected from solution onto a centrifugal filter membrane (0.65 μm pore size, PVDF membrane, Millipore) and then washed with 3 mL of water to remove any excess NHS-LC-biotin. The particles were re-suspended in 2 mL of water.

### Example 17

### Synthesis of Acrylate-Based Anti-CD11b Targeted, Ova Peptide-Containing PRINT Particles

This is a non-degradable control for Example 16.

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 2×2×1 μm cubes. The PFPE-DMA covered master was then subjected to UV light (λ=365 nm) for 3 minutes while under a nitrogen purge. The fully cured PFPE-DMA mold was then released from the silicon master. Separately, a mixture of 67 mg trimethylolpropane ethoxylate triacrylate (˜900 MW), 20 mg PEG-diacrylate (PEG MW ˜400), 10 mg 2-aminoethylmethacrylate, 2 mg ova peptide (SIINFEKL, Anaspec, Inc.), 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm²) for 2 minutes while maintaining a nitrogen purge. The particles were harvested by placing 2 mL of chloroform on the mold and scrapping the surface with a glass slide. The particle suspension was transferred to a scintillation vial.

### Biotinylation

One mold of particles with the composition listed above was harvested into 9 mL of water. PBS (1 mL, 10×) was added followed by Sulfo-NHS-LC-Biotin (100 μL, 45 mM in DMSO) and the mixture was stirred for 0.5 h. Particles were collected from solution onto a centrifugal filter membrane (0.65 μm pore size, PVDF membrane, Millipore) and then washed with 3 mL of water to remove any excess NHS-LC-biotin. The particles were re-suspended in 2 mL of water.

### Streptavidination

Streptavidin AlexaFluor 647 (100 μL, 2 mg/mL in PBS, Invitrogen) was added to the particle solution from above. After stirring for 0.5 h, particles were again collected onto a centrifugal filter membrane (0.65 μm pore size, PVDF membrane, Millipore) and then washed with 1 mL of water to remove any unbound streptavidin. The particles were re-suspended in 0.5 mL of water.

### Conjugation of Biotinylated Antibodies

Anti-mouse CD11b (100 μL, 0.5 mg/mL in PBS, eBiosciences) was added to the aliquot of particles from above. The mixture was stirred for 0.5 h. Particles were collected onto a centrifugal filter membrane (0.65 μm pore size, PVDF membrane, Millipore) and were washed with 1 mL of water to remove any unbound antibody. The particles were left dry on the filter membrane for delivery to prevent passive diffusion of the peptide cargo from the particle.

### Example 18

### Synthesis of Disulfide-Based, UltraAvidinated, Fluorescein-Containing Print Particles for T Cell Uptake Experiments

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 68 mg trimethylolpropane ethoxylate triacrylate (˜900 MW), 20 mg N,N-cystaminebisacrylamide, 10 mg 2-aminoethylmethacrylate, 1 mg fluorescein-o-acryalte, 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm2) for 2 minutes while maintaining a nitrogen purge. The particles were harvested by placing 2 mL of chloroform on the mold and scrapping the surface with a glass slide. The particle suspension was transferred to a scintillation vial.

### Biotinylation

Five molds of particles with the composition listed above were harvested into 17 mL of chloroform. NHS-PEO₁₂-Biotin (250 μL, 42 mg/mL in DMSO) was added and the mixture was stirred for 3.5 hours. Acetic anhydride (50 μl) was added and the mixture was stirred for 0.5 h. Particles were purified by vacuum filtration (P8, Fisherbrand) and collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water.

### Avidination

UltraAvidin (2 mL, 2.5 mg/mL in water) was added to the particle solution from above. After stirring for 18 hours, particles were collected onto a centrifugal filter membrane (0.1 μm pore size, PVDF membrane, Millipore) and then washed with 15 mL of water to remove any unbound avidin. The particles were re-suspended in 1.2 mL of water. The solution was analyzed by TGA, DLS, zeta potential, and SEM. The remaining particle solution was concentrated to 0.5 mL of a 2.8 mg/mL solution by centrifugation (12,000 rpm, 2 min). The particles were spun down into a pellet and the desired amount of supernatant removed.

### Titration of Available Biotin Binding Sites

Available biotin binding sites were quantified by fluorescence spectroscopy. UltraAvidinated PRINT particles (50 μL, 1.4 mg/mL) were added to a solution of biotin-4-fluorescein (90 nM in water) and the mixture was stirred for 10 min. Particles were removed from solution by filtration (0.1 μm pore size, PVDF membrane, Millipore) and the concentration of biotin-4-fluorescein remaining in solution was then determined. The decrease in concentration of biotin-4-fluorescein can be attributed to binding to UltraAvidinated PRINT particles (and thus removal from solution by filtration). Results translated to ˜5500 binding sites/particle based on an average particle weight of 6.91×10⁻¹⁵. The number of copies of UltraAvidin/particle was in the range 1,833-5,500 depending on the number of biotin binding sites occupied during attachment to the particle. The low value assumes 3 binding sites were occupied by attachment to the particle whereas the high number assumes only one site was used.

Disulfide-based, UltraAvidinated, fluorescein-containing PRINT particles used for T cell uptake experiments had a size of 360 nm (as determined by DLS), a PDI of 0.069 and a zeta potential of +9.0 mV.

### Example 19

### Synthesis of Disulfide-Based, UltraAvidinated, Dexamethasone-Containing Print Particles for T Cell Killing Experiments

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 67 mg trimethylolpropane ethoxylate triacrylate (˜900 MW), 20 mg N,N-cystaminebisacrylamide, 10 mg 2-aminoethylmethacrylate, 2 mg dexamethasone, 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm2) for 2 minutes while maintaining a nitrogen purge. The particles were harvested by placing 2 mL of chloroform on the mold and scrapping the surface with a glass slide. The particle suspension was transferred to a scintillation vial.

### Biotinylation

Five molds of particles with the composition listed above were harvested into 16 mL of chloroform. NHS-PEO₁₂-Biotin (250 μL, 50 mg/mL in DMSO) was added and the mixture was stirred for 20 hours. Acetic anhydride (200 μL) was added and the mixture was stirred for 1 h. Particles were collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water. DMSO (2 mL) was then added.

### Avidination

UltraAvidin (1 mL, 2.5 mg/mL in water) was added to the particle solution from above. After stirring for 18 hours, particles were diluted with 10 mL of water, purified by vacuum filtration (P8, Fisherbrand), and pelleted from the filtrate using centrifugation (8500 rpm, 50 mL falcon tube). The supernatant was removed and the particles were re-suspended in 30 mL of water. The particles were again pelleted and the supernatant removed leaving approximately 5 mL of water. Particles were collected from the remaining solution onto a centrifugal filter membrane (0.1 μm pore size, PVDF membrane, Millipore) and then washed with 15 mL of water to remove any unbound avidin. The particles were re-suspended in 1.2 mL of water. The solution was analyzed by TGA, DLS, zeta potential, and SEM. The remaining particle solution was concentrated to 0.5 mL of a 2.3 mg/mL solution by centrifugation (10,000 rpm, 2 min). The particles were spun down into a pellet and the desired amount of supernatant removed.

UltraAvidinated PRINT particles containing dexamethasone had a size of 329 nm (as determined by DLS), a PDI of 0.278 and a zeta potential of −29.0 mV.

### Example 20

### Synthesis of Disulfide-Based, UltraAvidinated, Print Particles for T Cell Killing Experiments

This is a negative control for Example 19.

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 69 mg trimethylolpropane ethoxylate triacrylate (˜900 MW), 20 mg N,N-cystaminebisacrylamide, 10 mg 2-aminoethylmethacrylate, 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm²) for 2 minutes while maintaining a nitrogen purge. The particles were harvested by placing 2 mL of chloroform on the mold and scrapping the surface with a glass slide. The particle suspension was transferred to a scintillation vial.

### Biotinylation

Five molds of particles with the composition listed above were harvested into 16 mL of chloroform. NHS-PEO₁₂-Biotin (250 μL, 50 mg/mL in DMSO) was added and the mixture was stirred for 20 hours. Acetic anhydride (200 μL) was added and the mixture was stirred for 1 h. Particles were collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water. DMSO (2 mL) was then added.

### Avidination

UltraAvidin (1 mL, 2.5 mg/mL in water) was added to the particle solution from above. After stirring for 18 hours, particles were diluted with 10 mL of water, purified by vacuum filtration (P8, Fisherbrand), and pelleted from the filtrate using centrifugation (8500 rpm, 50 mL falcon tube). The supernatant was removed and the particles were re-suspended in 30 mL of water. The particles were again pelleted and the supernatant removed leaving approximately 5 mL of water. Particles were collected from the remaining solution onto a centrifugal filter membrane (0.1 μm pore size, PVDF membrane, Millipore) and then washed with 15 mL of water to remove any unbound avidin. The particles were re-suspended in 1.2 mL of water. The solution was analyzed by TGA, DLS, zeta potential, and SEM. The remaining particle solution was concentrated to 0.5 mL of a 2.3 mg/mL solution by centrifugation (10,000 rpm, 2 min). The particles were spun down into a pellet and the desired amount of supernatant removed.

### Titration of Available Biotin Binding Sites

Available biotin binding sites were quantified by fluorescence spectroscopy. UltraAvidinated PRINT particles (25 μL, 1.2 mg/mL) were added to a solution of biotin-4-fluorescein (102 nM in water) and the mixture was stirred for 10 min. Particles were removed from solution by filtration (0.1 μm pore size, PVDF membrane, Millipore) and the concentration of biotin-4-fluorescein remaining in solution was then determined. The decrease in concentration of biotin-4-fluorescein can be attributed to binding to UltraAvidinated PRINT particles (and thus removal from solution by filtration). Results translated to ˜6700 binding sites/particle based on an average particle weight of 6.91×10⁻¹⁵.

Ultraavidinated, disulfide-based PRINT particles had a size of 384 nm (as determined by DLS), a PDI of 0.080 and a zeta potential of −23.4 mV.

### Example 21

### Synthesis of Disulfide-Based, UltraAvidinated, Doxorubicin-Containing Print Particles for T Cell Killing Experiments

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 67 mg trimethylolpropane ethoxylate triacrylate (˜900 MW), 20 mg N,N-cystaminebisacrylamide, 10 mg 2-aminoethylmethacrylate, 2 mg doxorubicin HCl, 1 mg hydroxycyclohexylphenylketone was prepared by dissolving the last four components in 50 μL DMSO and then adding the trimethylolpropane ethoxylate triacrylate. This mixture was spotted directly onto the patterned PFPE-DMA mold and covered with an un-patterned raw PET film. The monomer mixture was pressed between the two polymer sheets, and spread using a roller. The mold and PET sheet were then passed through a heated laminator (45° C., 10 cm/min). The mold was delaminated as it came out of the laminator. The mold was then subjected to UV light (λ=365 nm, power >20 mW/cm²) for 2 minutes while maintaining a nitrogen purge. The particles were harvested by placing 2 mL of chloroform on the mold and scrapping the surface with a glass slide. The particle suspension was transferred to a scintillation vial.

### Biotinylation

Seven molds of particles with the composition listed above were harvested into 20 mL of chloroform. Particles were collected from solution onto a centrifugal filter membrane (0.1 μm pore size, PVDF membrane, Millipore), and then washed with ˜20 mL chloroform to remove any surface-adhered dox. The particles were re-suspended in 10 mL chloroform and NHS-PEO₁₂-Biotin (500 μL, 25 mg/mL in DMSO) was added. The mixture was stirred for 5 hours. Acetic anhydride (200 μL) was then added and the mixture was stirred for an additional 0.5 h. Particles were collected from solution onto a centrifugal filter membrane (0.1 μm pore size, PVDF membrane, Millipore), re-suspended in ultra pure water, stirred for 5 min and collected again on a new centrifugal filter membrane (0.1 μm pore size, PVDF membrane, Millipore) to remove excess NHS-PEO₁₂-Biotin and acetic anhydride. The particles were re-suspended in 5 mL ultra pure water.

### Avidination

UltraAvidin (1 mL, 2.5 mg/mL in water) was added to the particle solution from above. After stirring for 14 hours, particles were collected onto a centrifugal filter membrane (0.1 μm pore size, PVDF membrane, Millipore) and then washed with 10 mL of water to remove any unbound avidin. The particles were re-suspended in 10 mL of water, purified by vacuum filtration (P8, Fisherbrand), and collected from solution onto a centrifugal filter membrane (0.1 μm pore size, PVDF membrane, Millipore). The particles were re-suspended in 1.1 mL of ultra pure water.

UltraAvidinated, dox-loaded PRINT particles had a size of 346 nm (as determined by DLS), a PDI of 0.266 and a zeta potential of −17.9 mV.

### Example 22

### Synthesis of Pro-Drug, UltraAvidinated, Doxorubicin-Containing Print Particles for T Cell Killing Experiments

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 50 mg PEG₂₀₀-CI-methacrylate (see Example 9), 49 mg trimethylolpropane ethoxylate triacrylate (˜428 MW), 1 mg 2,2-diethoxyacetophenone was prepared. This mixture was spotted directly onto the patterned PFPE-DMA mold and covered with a polyethylene (PE) film. The monomer mixture was pressed between the two polymer sheets, and spread using a roller. The PE sheet was then slowly peeled from the mold and then subjected to UV light (λ=365 nm) for 2 minutes while maintaining a nitrogen purge.

### Particle Synthesis

Twenty-eight molds of particles with the composition listed above were PRINTed by spotting monomer onto the molds (90:10 solids:solvent in DMSO), spreading the monomer under a poly(ethylene) sheet (using a roller), and slowly peeling away the sheet. The particles were harvested into filtered acetone and pelleted by ultracentrifugation. The solvent was removed, and the pellet dried under vacuum yielding 13.5 mg of a white solid.

### Hydrazide/Biotin Functionalization

CI-functionalized particles (8.0 mg, 1 eq. CI groups total) were suspended in filtered DMF (4 mL) in a glass scintillation vial. A DMF solution (660 μL) containing β-Alanyl (BOC)hydrazide (1.2 mg, 2 eq.; King et al., Bioconjugate Chem. 10:279-88, 1999), NH₂(PEO)-biotin (10.8 mg, 2 eq., MW=750), and NEt₃ (2.8 μL, 4 eq.) was then added to the particle solution and the mixture was stirred for 20 hours at rt. The particles were pelleted by ultracentrifugation, and the solvent removed. The particles were then washed repeatedly to remove any un-reacted starting materials (first with DMF—5×1 mL, then anhydrous methanol—5×1 mL). The pellet was dried under vacuum yielding 8 mg of a white solid.

### Hydrazide De-Protection, Dox Binding, and Avidination

Hydrazide functionalized particles (4.0 mg, 1 eq. CI groups total) were dissolved in a CH₂Cl₂:TFA solution (1:1, 2 mL) and stirred for 2.5 hours. The solvent was removed by rotovap. The particles were re-suspended in 2 mL anhydrous methanol and dox HCl (7.4 mg in 0.5 mL anhydrous methanol, 5 eq.) was added. The particles were stirred for 22 hours at 50° C. Particles were collected from solution by pelleting via centrifugation. They were washed extensively with anhydrous methanol (3×10 mL), and then PBS (1×1 mL), pelleted, and re-suspended in 2.5 mL of filtered PBS. UltraAvidin (1.2 mL, 2.5 mg/mL in PBS) was added and the particles were stirred for 18 hours at rt. Particles were diluted to 15 mL with HPLC grade water, filtered through Fisherbrand P8 filter paper. An additional 15 mL of HPLC grade water was then passed through the filter paper. Particles were pelleted from the filtrate via ultracentifugation. They were then re-suspended in 5 mL of water and collected on a centrifugal filter membrane (0.1 μm pore size, PVDF membrane, Millipore), washed with an additional 10 mL of water and finally re-suspended in 2.5 mL of HPLC grade water (1.4 mg/mL by TGA). A 0.5 mL aliquot was removed for analysis and 200 μL of 10×PBS was added to the remaining particle solution.

### Measurement of Avidin Surface Concentration

The amount of avidin bound to the particle surface was measured by titration of biotin binding sites with biotin-4-fluorescein. A PRINT particle solution (50 μL of a 1.4 mg/mL solution in water) was diluted to a final volume of 2.0 mL with PBS, and then titrated with 1.16×10⁻⁷ M biotin-4-fluorescein. The fluorescence of the fluorescein tag (ex. 492, em. 525) was monitored. The fluorescence of the fluorescein tag begins to increase linearly after the addition of approximately 780 μl of biotin. Assuming the mass of a single 200 nm PRINT particle to be 6.91×10⁻¹⁵ g, there were ˜5400 biotin binding sites per particle.

UltraAvidinated, hydrazone-linked dox PRINT particles had a size of 367 nm (as determined by DLS), a PDI of 0.207 and a zeta potential of −12.5 mV. The hydrazone linker in these particles is pH sensitive and is stable at pH 7.0, but degrades rapidly when the pH is lowered to 5-6.

### Example 23

### Synthesis of Pro-Drug, UltraAvidinated, Acetone-Containing Print Particles for T Cell Killing Experiments

This is a negative control for Example 22.

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 50 mg PEG₂₀₀-CI-methacrylate (see Example 9), 49 mg trimethylolpropane ethoxylate triacrylate (˜428 MW), 1 mg 2,2-diethoxyacetophenone was prepared. This mixture was spotted directly onto the patterned PFPE-DMA mold and covered with a polyethylene (PE) film. The monomer mixture was pressed between the two polymer sheets, and spread using a roller. The PE sheet was then slowly peeled from the mold and then subjected to UV light (λ=365 nm) for 2 minutes while maintaining a nitrogen purge.

### Particle Synthesis

Twenty-eight molds of particles with the composition listed above were PRINTed by spotting monomer onto the molds (90:10 solids:solvent in DMSO), spreading the monomer under a poly(ethylene) sheet (using a roller), and slowly peeling away the sheet. The particles were harvested into filtered acetone and pelleted by ultracentrifugation. The solvent was removed, and the pellet dried under vacuum yielding 13.5 mg of a white solid.

### Hydrazide De-protection, Acetone Binding, and Avidination

Hydrazide functionalized particles (4.0 mg, 1 eq. CI groups total) were dissolved in a CH₂Cl₂:TFA solution (1:1, 2 mL) and stirred for 2.5 hours. The solvent was removed by rotovap. The particles were re-suspended in 2 mL anhydrous methanol and acetone (500 μL of a 1.58 mg/mL solution in methanol, 0.79 mg, 5 eq.) was added. The particles were stirred for 22 hours at 50° C. Particles were collected from solution by pelleting via centrifugation. They were washed extensively with anhydrous methanol (3×10 mL), and then PBS (1×1 mL), pelleted, and re-suspended in 2.5 mL of filtered PBS. UltraAvidin (1.2 mL, 2.5 mg/mL in PBS) was added and the particles were stirred for 18 hours at rt. Particles were diluted to 15 mL with HPLC grade water, filtered through Fisherbrand P8 filter paper. An additional 15 mL of HPLC grade water was then passed through the filter paper. Particles were pelleted from the filtrate via ultracentifugation. They were then re-suspended in 5 mL of water and collected on a centrifugal filter membrane (0.1 μm pore size, PVDF membrane, Millipore), washed with an additional 10 mL of water and finally re-suspended in 2.5 mL of HPLC grade water (0.9 mg/mL by TGA). A 0.5 mL aliquot was removed for analysis and 200 μL of 10×PBS was added to the remaining particle solution.

### Measurement of Avidin Surface Concentration

The amount of avidin bound to the particle surface was measured by titration of biotin binding sites with biotin-4-fluorescein. A PRINT particle solution (75 μl of a 0.9 mg/mL solution in water) was diluted to a final volume of 2.0 mL with PBS, and then titrated with 1.16×10⁻⁷ M biotin-4-fluorescein. The fluorescence of the fluorescein tag (ex. 492, em. 525) was monitored. The fluorescence of the fluorescein tag begins to increase linearly after the addition of approximately 960 μL of biotin. Assuming the mass of a single 200 nm PRINT particle to be 6.91×10⁻¹⁵ g, there were ˜6700 biotin binding sites per particle.

UltraAvidinated, hydrazone-linked acetone PRINT particles had a size of 379 nm (as determined by DLS), a PDI of 0.245 and a zeta potential of −2.3 mV.

### Example 24

### Synthesis of UltraAvidinated, Dox-Containing Print Particles for T cell killing experiments

This is a non-degradable control for Example 22.

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 50 mg PEG₂₀₀-CI-methacrylate (see Example 9), 49 mg trimethylolpropane ethoxylate triacrylate (˜428 MW), 1 mg 2,2-diethoxyacetophenone was prepared. This mixture was spotted directly onto the patterned PFPE-DMA mold and covered with a polyethylene (PE) film. The monomer mixture was pressed between the two polymer sheets, and spread using a roller. The PE sheet was then slowly peeled from the mold and then subjected to UV light (λ=365 nm) for 2 minutes while maintaining a nitrogen purge.

### Particle Synthesis

Twenty-eight molds of particles with the composition listed above were PRINTed by spotting monomer onto the molds (90:10 solids:solvent in DMSO), spreading the monomer under a poly(ethylene) sheet (using a roller), and slowly peeling away the sheet. The particles were harvested into filtered acetone and pelleted by ultracentrifugation. The solvent was removed, and the pellet dried under vacuum yielding 13.5 mg of a white solid.

### Conjugation of Dox/Biotin (Via Non-Degradable Amide Bond), Avidination

CI-functionalized particles (4.0 mg, 1 eq. CI groups total) were suspended in filtered DMF (2 mL) in a glass scintillation vial. A DMF solution (330 μL) containing Doxorubicin HCl (1.6 mg, 2 eq.), NH₂(PEO)-biotin (5.4 mg, 2 eq., MW=750), and NEt₃ (1.4 μL, 4 eq.) was then added to the particle solution and the mixture was stirred for 20 hours at rt. The particles were pelleted by ultracentrifugation, and the solvent removed. The particles were then washed repeatedly to remove any un-reacted starting materials (first with DMF—5×1 mL, then anhydrous methanol—5×1 mL). The pellet was dried under vacuum yielding 4 mg of a pink solid. Particles were re-suspended in 2.5 mL PBS. UltraAvidin (1.2 mL, 2.5 mg/mL in PBS) was added and the particles were stirred for 18 hours at rt. Particles were diluted to 15 mL with HPLC grade water, filtered through Fisherbrand P8 filter paper. An additional 15 mL of HPLC grade water was then passed through the filter paper. Particles were pelleted from the filtrate via ultracentifugation. They were then re-suspended in 5 mL of water and collected on a centrifugal filter membrane (0.1 μm pore size, PVDF membrane, Millipore), washed with an additional 10 mL of water and finally re-suspended in 2.5 mL of HPLC grade water (1.5 mg/mL by TGA). A 0.5 mL aliquot was removed for analysis and 200 μL of 10×PBS was added to the remaining particle solution.

### Measurement of Avidin Surface Concentration

The amount of avidin bound to the particle surface was measured by titration of biotin binding sites with biotin-4-fluorescein. A PRINT particle solution (25 μl of a 1.5 mg/mL solution in water) was diluted to a final volume of 2.0 mL with PBS, and then titrated with 1.16×10⁻⁷ M biotin-4-fluorescein. The fluorescence of the fluorescein tag (ex. 492, em. 525) was monitored. The fluorescence of the fluorescein tag begins to increase linearly after the addition of approximately 440 μL of biotin. Assuming the mass of a single 200 nm PRINT particle to be 6.91×10⁻¹⁵ g, there were ˜5700 biotin binding sites per particle.

UltraAvidinated, carbamate-linked dox PRINT particles had a size of 413 nm (as determined by DLS), a PDI of 0.277 and a zeta potential of −3.9 mV. The dox is conjugated through a stable carbamate bond.

### Example 25

### Synthesis of Pro-Drug, Hydrazone-Linked Dox Print Particles for Dox Release Experiments by Flow Cytometry

### Particle Fabrication

A patterned perfluoropolyether (PFPE) mold was generated by pouring a PFPE-dimethacrylate (PFPE-DMA) containing 1-hydroxycyclohexyl phenyl ketone over a silicon substrate patterned with 1000 nm tall×1000 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 50 mg PEG₂₀₀-CI-methacrylate (see Example 9), 49 mg trimethylolpropane ethoxylate triacrylate (˜428 MW), 1 mg 2,2-diethoxyacetophenone was prepared. This mixture was spotted directly onto the patterned PFPE-DMA mold and covered with a polyethylene (PE) film. The monomer mixture was pressed between the two polymer sheets, and spread using a roller. The PE sheet was then slowly peeled from the mold and then subjected to UV light (λ=365 nm) for 2 minutes while maintaining a nitrogen purge.

### Particle Synthesis

Eight molds of particles with the composition listed above were PRINTed by spotting monomer onto the molds (90:10 solids:solvent in DMSO), spreading the monomer under a poly(ethylene) sheet (using a roller), and slowly peeling away the sheet. The particles were harvested into filtered acetone and pelleted by ultracentrifugation. The solvent was removed, and the pellet dried under vacuum yielding ˜12 mg of a white solid.

### Hydrazide Functionalization

CI-functionalized particles (8 mg, 1 eq. CI groups total) were suspended in DMF. A DMF solution (1 mL) containing β-Alanyl(BOC)hydrazide (38 mg, 20 eq.) and NEt₃ (52 μL, 40 eq.) was then added to the particle solution and the mixture was stirred for 20 hours at rt. The particles were pelleted by ultracentrifugation, and the solvent removed. The particles were then washed repeatedly to remove any un-reacted starting materials (first with DMF—1×5 mL, then anhydrous methanol—3×5 mL). The pellet was dried under vacuum.

### Hydrazide De-Protection and Dox Binding

Hydrazide functionalized particles from above (4.0 mg, 1 eq. CI groups total) were dissolved in a CH₂Cl₂:TFA solution (1:1, 2 mL) and stirred for 2.5 hours. The solvent was removed by rotovap. The particles were re-suspended in 2 mL anhydrous methanol and dox HCl (6.5 mg in 1.5 mL anhydrous methanol, 2.4 eq.) was added. The particles were stirred for 20 hours at 50° C. Particles were collected from solution by pelleting via centrifugation. They were washed extensively with anhydrous methanol (4×5 mL) and then PBS (1×5 mL), re-suspended in PBS (4 mL) and stirred for 3 hours at rt. Particles were then pelleted, washed with anhydrous methanol (2×1 mL) and dried under vacuum leaving 2.9 mg of a slightly pink solid.

### Dox Release by Flow Cytometry

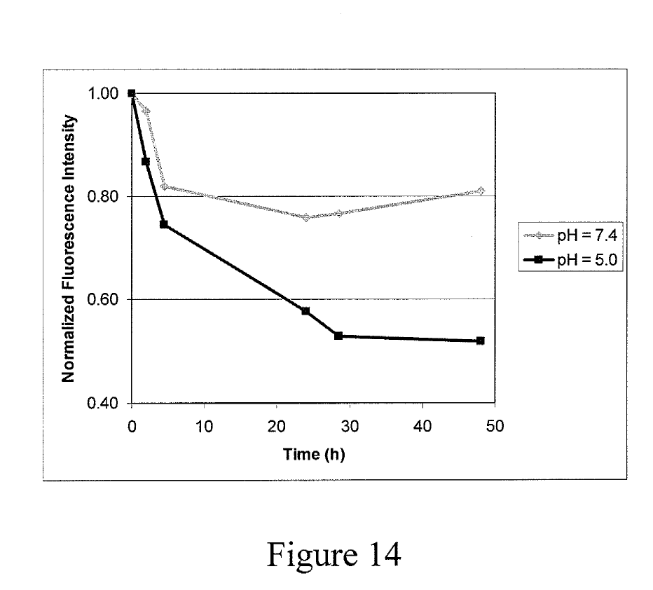

Fluorescence intensity of pro-drug, avidinated, dox-loaded PRINT particles stirred in buffered solutions (pH=5.0 and 7.4) at 37° C. for 48 hours was monitored by flow cytometry (FIG. 14). Dox release was strongly dependent on pH. There appeared to be some burst release in both samples likely due to dox adsorbed to the surface of the particles. After the initially burst release, the particles look stable at a pH of 7 at slightly elevated temperature (they should only release small amounts of dox if stored at 4° C. at pH=7.4). The particles stirred at pH=5.0 appeared to release only about half of their dox; however, the particles also became less disperse over time.

Pro-drug, hydrazone-conjugated dox PRINT particles had a size of 1128 nm (as determined by DLS) and a PDI of 0.196.

### Example 26

### Targeted Cell Killing of Sup-B8 Cancer Cells Using Pro-Drug, UltraAvidinated, Doxorubicin-Containing PRINT Particles

### Particle Synthesis

Samples of particles described in Examples 22-24 (250 g particles each) were stirred with biotin-PEG-peptide (10 eq. to ultraavidin) for 2 hours at rt and then concentrated to 5 mg/mL via centrifugation. Sup-B8 (targeted) or Ramos cells (non-targeted) were collected and washed with OPTI-MEM I/GlutaMAX medium supplemented with non-essential amino acids. Then cells were plated in 96 well flat bottom plate at 200,000 per well, and were mixed with various amount of particles and dosed at 37° C. for 2 hours.

### Cell Uptake Assay

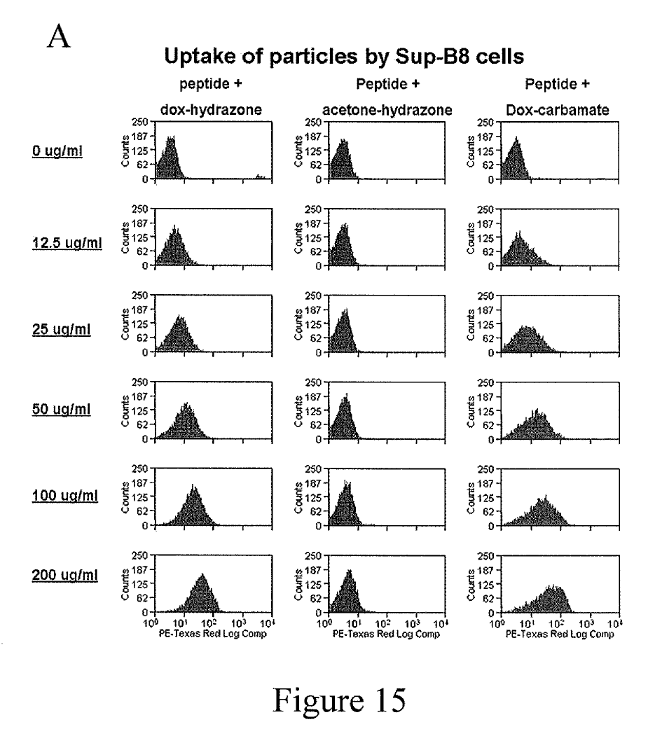

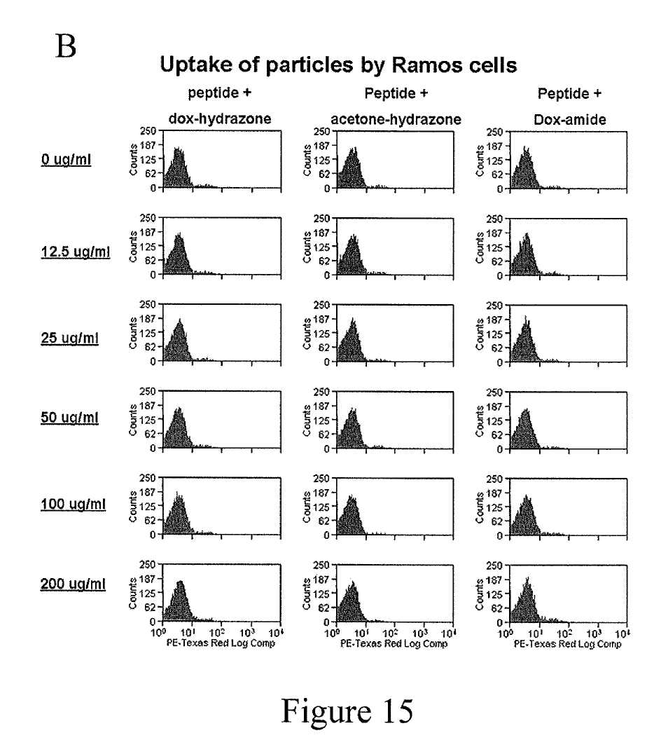

Cells were transferred to 96 well round bottom plate and chilled on ice for 5 min. Then they were spun down at 1,200 rpm for 5 min at 4° C., and medium was removed. 100 ul of ice cold acid buffer (200 mM acetic acid and 0.5M NaCl) was added to each well and kept on ice for 5 min. Then cells were spun down at 1,200 rpm for 5 min at 4° C. followed by two washes with ice cold FACS wash buffer (DPBS, 2% FBS, 0.1% NaN3). Finally cells were resuspended in 200 ul FACS fixation buffer (DPBS, 1% paraformaldehyde) and analyzed on Cyan ADP analyzer. Results are shown in FIG. 15.

### Cell Viability Assay

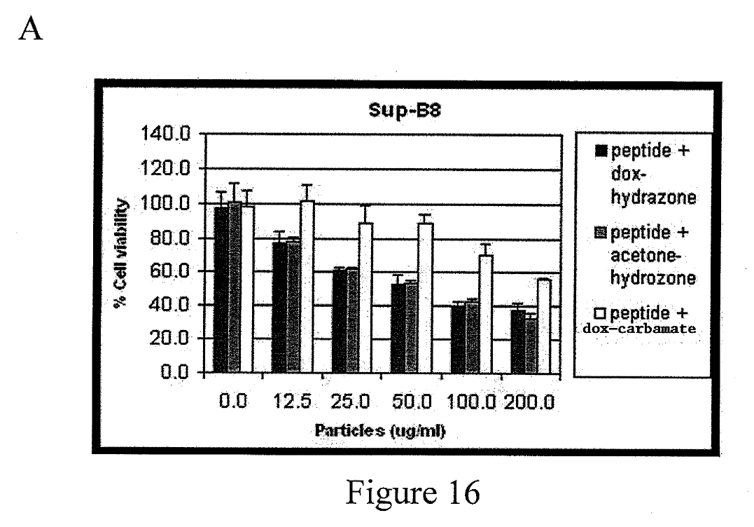

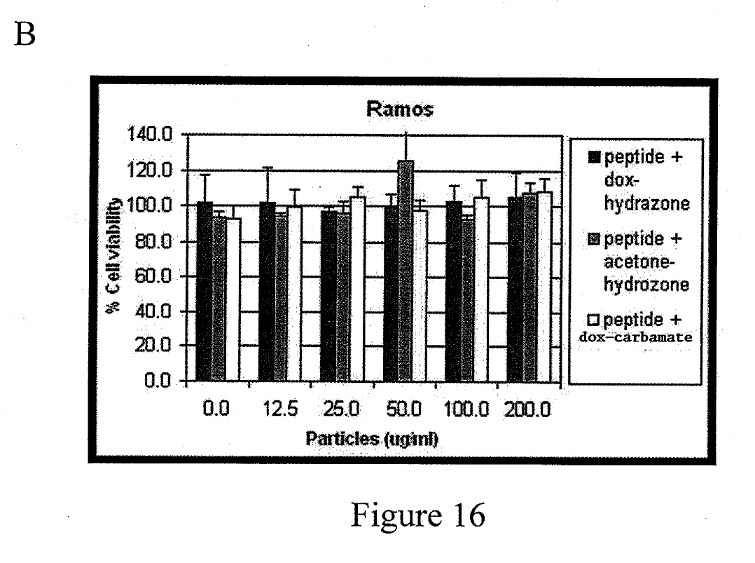

Cells were spun down at 1,200 rpm for 5 min and media were removed. Replaced with 200 ul fresh complete medium (RPMI 1640 w/10% FBS) per well. Cells were kept at 37° C. CO2 incubator for 72 hours. Right before the assay replaced with 100 ul fresh complete medium. Added 20 ul Cell titer 96 Aqueous one solution cell viability assay reagent (Promega) to each well and incubated at 37 C for about 1 h. Collected absorptions at OD490 with plate reader. Results are shown in FIG. 16.

### Example 27

### Targeting T Cells Using Disulfide-Based Cylindrical Particles with MHC-Antigen/Avidin-Functionalized Surfaces Containing Fluorescein-o-Acrylate

Avidinated PRINT particles (41.67 μL) generated in Example 18 were incubated with NRPV7-Kd (3.28 μL of a 2.08 μg/μL solution) or HA-Kd (2.44 μL of a 2.88 μg/μL solution) on ice for 30 min followed by quenching of remaining biotin binding sites with 5 μl of 500 μM biotin solution. The particles were incubated on ice for an additional 10 min.

### MHC Preparation

Bacteria were transformed with a MHC I plasmid coding for the D^{b} MHC I molecules with a 15 amino acid tag (GLNDIFEAQKIEWHE). The tag conferred the ability to biotinylate the protein with the enzyme BirA. The bacteria were grown in 13 liters of selective medium. The bacteria were pelleted and passed through a French Press at 16,000 psi, and the MHC molecules isolated from the inclusion bodies in 8M urea. The MHC I molecules were refolded with the peptide of interest in the presence of beta-2-microglobulin for 24-36 hours at 10 degrees C. in 1 liter of Refolding buffer (Refolding buffer=100 mM Tris (pH=8.0), 400 mM L-Arginine, 2 nM EDTA, 1 ug/ml leupeptin, 2 ug/ml aprotinin, 4.9 nM GSH (glutathione reduced), 0.49 mM GSSH (glutathione oxidized). The refolded MHC/peptide/beta2M complexes were concentrated to 25 ml in a nitrogen pressure filtration device with a 10,000 MWCO filter and then concentrated to 1 ml in a Centricon with a 10,000 MWCO filter. The MHC/peptide/beta2M was then purified through a HPLC column.

The spleens from one NOD-CL4 mouse and one NOD-8.3 mouse were isolated and disassociated. The cells were resuspended in Ammonium chloride Red Blood Cell Lysis Buffer (0.15 M NH₄Cl, 1. M KHCO₃, 0.1 mM Na₂EDTA, pH to 7.2-7.4), washed 2 times, and strained (Falcon # 352340) and counted. The spleenocytes were blocked in FcR Block (24G2, B-cell hybridoma supernatant) for 20 minutes on ice, at a concentration of 1×10⁶ cells per 10 μL of FcBlock. Antibodies were added in an additional volume of 40 μL, and PRINT particles were added in an additional 50 μL, for a total volume of 100 μL (dilutions were done in FACS WASH (=2% FBS, 0.1% NaN₃, PBS)). The antibodies and PRINT particles were incubated with the spleenocytes on ice and in the dark for an additional 25 minutes, and then washed 3 times in FACS WASH prior to analysis on the Cyan FACS machine.

The in vitro study used spleen cell populations from 8.3-NOD mice and CL4-NOD mice. In the 8.3 NOD mouse, 75% of their CD8+ T cells are transgenic and will recognize the NRP-V7 MHCI (Kd) tetramer. In the CL4-NOD mice, almost all of their CD8+ T cells are transgenic and will recognize the HA-MHCI (Kd) tetramer. The NRP-V7-Kd coated PRINT particles were found to target around 75% of the CD8+ T cells in the spleen of the 8.3-NOD mice but only 4% of the CD8+ T cells in the spleen of the CL4-NOD mice. The HA-Kd coated PRINT particles targeted 94% of the CD8+ T cells in the spleen of the HA-NOD mice but only 1% of the CD8+ T cells in the spleen of the 8.3-NOD mice.

### Example 28

### Targeting T Cell Killing Using Disulfide-Based Cylindrical Particles with MHC-Antigen/Avidin-Functionalized Surfaces Containing Doxorubicin

Avidinated PRINT particles (41.67 μL) generated in Example 21 were incubated with NRPV7-Kd (3.28 μL of a 2.08 μg/μL solution) or HA-Kd (2.44 μL of a 2.88 μg/μL solution) on ice for 30 min followed by quenching of remaining biotin binding sites with 5 μl of 500 μM biotin solution. The particles were incubated on ice for an additional 10 min.

### MHC Preparation

Bacteria were transformed with a MHC I plasmid coding for the D^{b} MHC I molecules with a 15 amino acid tag (GLNDIFEAQKIEWHE). The tag conferred the ability to biotinylate the protein with the enzyme BirA. The bacteria were grown in 13 liters of selective medium. The bacteria were pelleted and passed through a French Press at 16,000 psi, and the MHC molecules isolated from the inclusion bodies in 8M urea. The MHC I molecules were refolded with the peptide of interest in the presence of beta-2-microglobulin for 24-36 hours at 10 degrees C. in 1 liter of Refolding buffer (Refolding buffer=100 mM Tris (pH=8.0), 400 mM L-Arginine, 2 nM EDTA, 1 ug/ml leupeptin, 2 ug/ml aprotinin, 4.9 nM GSH (glutathione reduced), 0.49 mM GSSH (glutathione oxidized). The refolded MHC/peptide/beta2M complexes were concentrated to 25 ml in a nitrogen pressure filtration device with a 10,000 MWCO filter and then concentrated to 1 ml in a Centricon with a 10,000 MWCO filter. The MHC/peptide/beta2M was then purified through a HPLC column.

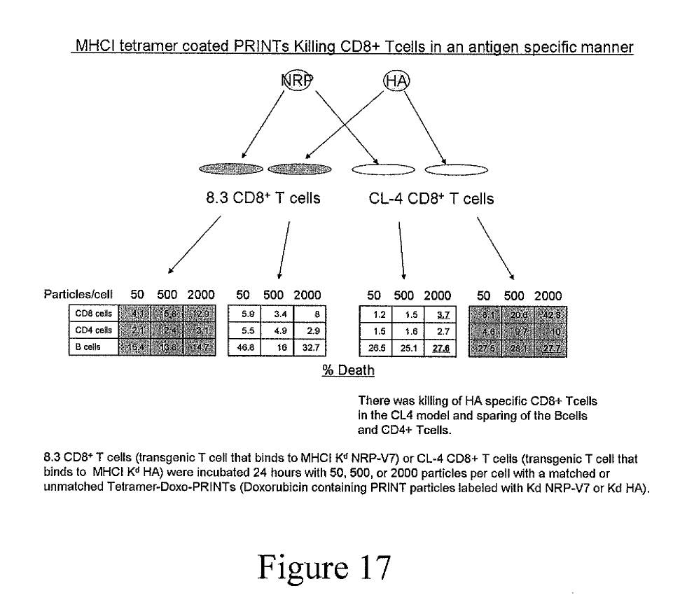

The spleens from one NOD-CL4 mouse and one NOD-8.3 mouse were isolated and disassociated. The cells were resuspended in Ammonium chloride Red Blood Cell Lysis Buffer (0.15 M NH₄Cl, 1. M KHCO₃, 0.1 mM Na₂EDTA, pH to 7.2-7.4), washed 2 times, and strained (Falcon # 352340) and counted. The spleenocytes were blocked in FcR Block (24G2, B-cell hybridoma supernatant) for 20 minutes on ice, at a concentration of 1×10⁶ cells per 10 μL of FcBlock. Antibodies were added in an additional volume of 40 μL, and PRINT particles were added in an additional 50 μL, for a total volume of 100 μL. The antibodies and PRINT particles were incubated with the spleenocytes for 24 hours in the presence of IL-7 to minimize basal T cell death. A cell viability assay was then performed (FIG. 17).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A therapeutic composition for eliciting an immune response, comprising a plurality of monodisperse micro and/or nanoparticles said particles having a predetermined geometry and a broadest dimension less than about 10 μm, wherein said particles comprise an immunogenic component and an antigen presenting cell (APC) targeting component.

2. The therapeutic composition of claim 1, wherein the immunogenic component is a peptide or a protein.

3. The therapeutic composition of claim 1, wherein the immunogenic component is derived from an infectious organism.

4. The therapeutic composition of claim 3, wherein the immunogenic component comprises a moiety selected from the group consisting of tetanus toxoid, influenza HA antigen, influenza NP antigen, an HIV antigen, a Hepatitis B antigen, a Hepatitis C antigen, diphtheria toxoid, a HPV antigen, a FeLV antigen, a parvovirus antigen, a distemper antigen, and combinations thereof.

5. The therapeutic composition of claim 1, wherein the immunogenic component comprises a tumor antigen.

6. The therapeutic composition of claim 5, wherein the tumor antigen is selected from the group consisting of an activated oncogene product, a tumor suppressor gene product, a reactivated embryonic gene product, a tissue specific differentiation antigen, a self protein, a viral gene product, an idiotypic epitope, and combinations thereof.

7. The therapeutic composition of claim 1, wherein the APC targeting component comprises a moiety selected from the group consisting of a polypeptide, a protein, a single-stranded nucleic acid, a double-stranded nucleic acid, a small molecule, and combinations thereof.

8. The therapeutic composition of claim 7, wherein the polypeptide comprises an antibody or an antibody fragment.

9. The therapeutic composition of claim 1, wherein the APC targeting component comprises a moiety selected from the group consisting of a CD11c ligand, a CD11b ligand, a CD11a ligand, granulocyte macrophage colony-stimulating factor (GMCSF), a CD18 ligand, a CD19 ligand, a CD20 ligand, a CD40 ligand, a CD205 ligand, a CMKLR1 ligand, a CD209 ligand, a CD83 ligand, a CD80 ligand, a CD86 ligand, a CCR7 ligand, a CD273 ligand, a DEC-205 ligand, a Toll-like receptor (TLR) agonist, and combinations thereof.

10. The therapeutic composition of claim 9, wherein the TLR agonist is selected from the group consisting of a tri-acyl lipopeptide, lipoteichoic acid, a double-stranded RNA, a lipopolysaccharide, flagellin, a diacyl lipopeptide, an imidazoquinoline, and a CpG-containing nucleotide sequence.

11. The therapeutic composition of claim 1, wherein the APC targeting component is associated with an outside surface of the particle.

12-20. (canceled)

21. A method of eliciting an immune response in a subject, the method comprising administering to the subject a pharmaceutical composition comprising a therapeutically effective amount of a plurality of monodisperse micro and/or nanoparticles said particles having a predetermined geometry and a broadest dimension less than about 10 μm, wherein said particles comprise an immunogenic component and an antigen presenting cell (APC) targeting component.

22. (canceled)

23. The method of claim 21, wherein the immunogenic component is derived from an infectious organism.

24. The method of claim 21, wherein the immunogenic component comprises a tumor antigen.

25. The method of claim 21, wherein the APC targeting component comprises a moiety selected from the group consisting of a polypeptide, a protein, a single-stranded nucleic acid, a double-stranded nucleic acid, a small molecule, and combinations thereof.

26-34. (canceled)

35. A pharmaceutical composition, comprising a plurality of monodisperse micro and/or nanoparticles said particles having a predetermined geometry and a broadest dimension less than about 10 μm, wherein said particles comprise an active agent and an immune cell targeting component.

36. The pharmaceutical composition of claim 35, wherein the active agent is an anticancer agent.

37. The pharmaceutical composition of claim 35, wherein the immune cell targeting component comprises a moiety selected from the group consisting of a polypeptide, a protein, a single-stranded nucleic acid, a double-stranded nucleic acid, a small molecule, and combinations thereof.

38. The pharmaceutical composition of claim 37, wherein the polypeptide comprises an antibody or an antibody fragment.

39. The pharmaceutical composition of claim 38, wherein the antibody or antibody fragment comprises an antibody or antibody fragment that specifically binds CD3, CD4, CD8, CD19, CD20, or CD40.

40-54. (canceled)
